(19) United States
(12) Patent Application Publication
(10) Pub. No.: US 2025/0248678 A1
(43) Pub. Date: Aug. 7, 2025

(54) SYSTEMS, METHODS, AND MEDIUM FOR CALIBRATING A PHOTON COUNTING COMPUTED TOMOGRAPHY (PCCT) SYSTEM MODEL

(71) Applicant: UNITED IMAGING HEALTHCARE NORTH AMERICA, INC., Houston, TX (US)

(72) Inventors: Wenying WANG, Houston, TX (US); Jinglu MA, Shanghai (CN)

(73) Assignee: UNITED IMAGING HEALTHCARE NORTH AMERICA, INC., Houston, TX (US)

(21) Appl. No.: 18/931,099

(22) Filed: Oct. 30, 2024

Related U.S. Application Data
(60) Provisional application No. 63/549,438, filed on Feb. 2, 2024.

(51) Int. Cl. A61B 6/58; A61B 6/03; A61B 6/42

(52) U.S. Cl. CPC A61B 6/583; A61B 6/03; A61B 6/4241

(57) ABSTRACT

A method may include obtaining a plurality of sets of photon counting data, each set of the plurality of sets of photon counting data being acquired by a photon counting computed tomography (PCCT) device scanning one of a plurality of phantoms according to one or more scanning parameters; obtaining a photon counting detector (PCD) system model of the PCCT device; and calibrating the PCCT system model by determining, based on the plurality of sets of photon counting data, the scanning parameters and phantom parameters of the phantom corresponding to each set of the plurality of sets of photon counting data, model parameters of the PCCT system model, the calibrated PCCT system model representing a mapping relationship between photon counting data, one or more scanning parameters, and one or more parameters of one or more reference materials through the determined model parameters.

### CROSS-REFERENCE TO RELATED APPLICATIONS

This application claims priority to Provisional U.S. Patent Application No. 63/549,438 filed on Feb. 2, 2024, the entire contents of which are hereby incorporated by reference.

### TECHNICAL FIELD

The present disclosure relates to the field of photon counting technology, and in particular, to a system and a method for calibrating a photon counting computed tomography (PCCT) system model, and a system and a method for image reconstruction based on the calibrated PCCT system model.

### BACKGROUND

Photon counting computed tomography (PCCT) technology offers improved spatial resolution, lower noise, good spectral separation, and the potential for direct material decomposition in the projection domain. The PCCT technology has great potential in various diagnostic imaging tasks thanks to its advantages in low noise, quantitative accuracy, material discrimination capabilities, etc.

Despite the above promising properties, PCCT is still restricted from wide clinical applications because of the concern in high-flux performance and stability at high clinical through-put. The application of PCCT technology generally requires the analysis and modeling of a PCCT system. Therefore, it is desirable to provide a system for calibrating a PCCT system model to enhance the accuracy of the calibrated PCCT system model.

### SUMMARY

One or more embodiments of the present disclosure provide a system, comprising: at least one processor and at least one storage device. The at least one storage device may be configured to store computer instructions. The at least one processor may be configured to communicate with the at least one storage device to direct, when the computer instructions are executed, the system to: obtain a plurality of sets of photon counting data, each set of the plurality of sets of photon counting data being acquired by a photon counting computed tomography (PCCT) device scanning one of a plurality of phantoms according to one or more scanning parameters; obtain a photon counting detector (PCD) system model; and calibrate the PCCT system model by determining, based on the plurality of sets of photon counting data, the one or more scanning parameters, and a parameter of the phantom, one or more model parameters of the PCCT system model.

In some embodiments, the model parameters may be related to at least one procedure of X-ray generation, X-ray absorption, energy deposition, energy resolution, charge sharing, pulse pileup effect, or energy separation in a generation process of photon counting data.

In some embodiments, the PCCT system model may include a plurality of sub-models, and the plurality of sub-models include at least one of: an energy deposition sub-model representing a relationship between the energy deposition and the photon counting data, an energy resolution sub-model representing a relationship between the energy resolution and the photon counting data, a charge sharing sub-model representing a relationship between the charge sharing and the photon counting data, a pulse pileup sub-model representing a relationship between the pulse pileup and the photon counting data, or an energy separation sub-model representing a relationship between the energy separation and the photon counting data.

In some embodiments, the model parameters may include one or more groups, each group of the one or more groups of the model parameters corresponding to different pulse pileup orders under an energy bin.

In some embodiments, the calibrated PCCT system model may include one or more spectrum models each of which corresponds to one of one or more energy bins, each of the one or more spectrum models representing a mapping relationship between photon counting data under the energy bin, the scanning parameter, and the parameter of the reference material through a group of model parameters corresponding to the energy bin.

In some embodiments, calibrating the PCCT system model based on the plurality of sets of photon counting data including determining a group of model parameters corresponding to an energy bin according to operations may include: for one of the different pulse pileup orders under the energy bin, determining an initial value of a model parameter among the group of model parameters corresponding to the pulse pileup order; and determining a target value of the model parameter based on the initial value of the model parameter corresponding to the pulse pileup order.

In some embodiments, the system may perform an optimization of the model parameters based on the initial values of the model parameters corresponding to the pulse pileup order to determine the target value of the model parameter. The the optimization may include performing an iterative process including multiple iterations. In each iteration, based on a portion of a set of photon counting data under the energy bin, the parameters of the phantoms and the one or more scanning parameters corresponding to the photon counting data measurements, photon counting estimation may be determined through the spectrum model including the initial values of the model parameters. Based on an error between the portion of the set of the photon counting data under the energy bin and the photon counting estimation, a target optimization function may be constructed. The initial values of the model parameters may be updated based on the target optimization function to obtain updated values of the model parameters. The updated values of the model parameters may be designated as the initial values of the model parameters in a next iteration. In response to determining that a termination condition is satisfied, updated values of the model parameters may be designated as the target value of the model parameter.

In some embodiments, the determining an initial value of a model parameter corresponding to the pulse pileup order may include: determining, based on a reference set of photon counting data obtained under a scanning parameter satisfying a condition, a reference model parameter; and determining, based on the reference model parameter, the initial value of the model parameter corresponding to the pulse pileup order.

In some embodiments, the determining, based on the reference model parameter, the initial value of the model parameter corresponding to the pulse pileup order includes: in response to determining that the pulse pileup order is zero, determining the initial value of the model parameter corresponding to the pulse pileup order by normalizing the reference model parameter based on a reference tube current.

In some embodiments, the determining, based on the reference model parameter, the initial value of the model parameter corresponding to the pulse pileup order may include: in response to determining that the pulse pileup order is non-zero, determining an initial value of the model parameter corresponding to a pulse pileup order of zero by normalizing the reference model parameter based on a reference tube current; and determining, based on the initial value of the model parameter corresponding to the pulse pileup order being of zero, the initial value of the model parameter corresponding to the pulse pileup order being of non-zero.

One or more embodiments of the present disclosure provide a system, comprising: at least one processor and at least one storage device. The at least one storage device may be configured to store computer instructions; and the at least one processor is configured to communicate with the at least one storage device to direct, when the computer instructions are executed, the system to: obtain a set of photon counting data acquired by a PCCT device scanning a target subject according to a target scanning parameter; obtain a calibrated PCCT system model representing a mapping relationship between photon counting data, a scanning parameter, and one or more material parameters of one or more reference materials; determine, based on the set of photon counting data and the target scanning parameter of the target subject, one or more target material parameters corresponding to the target subject through the calibrated PCCT system model; and obtain, based on the one or more target material parameters corresponding to the subject, one or more reconstructed images related to the target subject.

In some embodiments, the determining, based on the set of photon counting data and the target scanning parameter of the target subject, the target material parameters corresponding to the target subject through the calibrated PCCT system model may include: determining, based on the set of photon counting data, the target scanning parameter of the subject, and attenuation coefficients of the one or more reference materials corresponding to the target subject, target thicknesses of the one or more reference materials corresponding to the target subject through the calibrated PCCT system model.

In some embodiments, the obtaining, based on the target material parameters of the reference materials corresponding to the target subject, reconstructed images related to the target subject may include: determining, based on the target thicknesses of the one or more reference materials, a material line integral of each of the reference materials corresponding to the target subject; and generating, based on the material line integral of each of the reference materials corresponding to the target subject, a material decomposition image of each of the reference materials corresponding to the target subject.

In some embodiments, the determining, based on the set of photon counting data and the target scanning parameter of the target subject, the target material parameters corresponding to the target subject through the calibrated PCCT system model may include: determining, based on the set of photon counting data, the target scanning parameter of the subject, and attenuation coefficients of the one or more reference materials corresponding to the target subject, a target material line integral corresponding to the target subject through the calibrated PCCT system model.

In some embodiments, the obtaining, based on the target material parameters corresponding to the subject, one or more reconstructed images related to the target subject may include: determining, based on the target material line integral corresponding to the target subject, material line integrals of the one or more reference materials corresponding to the target subject; and reconstructing a material decomposition image of the target subject based on one of the material line integrals of the one or more reference materials corresponding to the target subject.

In some embodiments, the determining, based on the set of photon counting data and the target scanning parameter of the target subject, the target material parameters corresponding to the target subject through the calibrated PCCT system model may include: constructing, based on the calibrated PCCT system model, a comparison table; and determining, based on the comparison table, the set of photon counting data, and the target scanning parameter of the target subject, the target material parameters corresponding to the target subject.

In some embodiments, the determining, based on the comparison table, the set of photon counting data, and the target scanning parameter of the target subject, the target material parameters corresponding to the target subject may include: determining, based on the set of photon counting data and the target scanning parameter, first photon counting data and second photon counting data from the comparison table; determining, based on the first photon counting data and the second photon counting data, a first material parameter and a second material parameter corresponding to the target subject, respectively; and determining, based on the first material parameter and the second material parameter, a target material parameter corresponding to the target subject through interpolation processing.

In some embodiments, the determining, based on the set of photon counting data and the target scanning parameter of the target subject, the target material parameters corresponding to the target subject through the calibrated PCCT system model may include: obtaining the target material parameters corresponding to the target subject by inputting the set of photon counting data and the target scanning parameter of the target subject into the calibrated PCCT system model.

In some embodiments, the obtaining a calibrated PCCT system model may include: obtaining a plurality of sets of photon counting data, each set of the plurality of sets of photon counting data being acquired by the PCCT device scanning one of a plurality of phantoms according to one or more scanning parameters; obtaining a PCCT system model; and calibrating the PCCT system model by determining, based on the plurality of sets of photon counting data, the one or more scanning parameters, and a parameter of the phantom, one or more model parameters of the PCCT system model.

In some embodiments, the set of photon counting data includes a plurality of subsets of photon counting data corresponding to different energy bins. The obtaining, based on the one or more target material parameters corresponding to the subject, one or more reconstructed images related to the target subject may include: determining, based on the plurality of subsets of photon counting data, a material line integral of each of the reference materials under the different energy bins; determining, based on the material line integrals of the reference materials under the different energy bins, projection data of at least one energy bin; and obtaining, based on the projection data of the at least one energy bin, a material decomposition image corresponding to the target subject.

In some embodiments, the calibrated PCCT system model representing a mapping relationship between photon counting data, a scanning parameter, and a parameter of a reference material through the determined model parameters.

One of the embodiments of the present disclosure provides calibration method for a PCCT system model, implemented by a processor, comprising: obtaining spectral measurements by scanning a phantom; wherein the scanning corresponds to preset scanning parameters, and the phantom corresponds to a preset material line-integral; and obtaining a calibrated PCCT system model by performing parameter fitting on a PCCT system model to be calibrated based on the spectral measurements, the preset scanning parameters, and the preset material line-integral, wherein model parameters of the calibrated PCCT system model include energy spectra formation parameters determined by the parameter fitting; and the calibrated PCCT system model represents a correlation between the photon counting data and the material line-integral.

### BRIEF DESCRIPTION OF THE DRAWINGS

The present disclosure will be further illustrated by way of exemplary embodiments, which will be described in detail with the accompanying drawings. These embodiments are non-limiting. In these embodiments, the same count indicates the same structure, wherein:

FIG. 1 is a schematic diagram illustrating an exemplary PCCT system according to some embodiments of the present disclosure;

FIG. 2 is a flowchart illustrating an exemplary calibration method for a PCCT system model according to some embodiments of the present disclosure;

FIG. 3 is a schematic diagram illustrating an exemplary process of generating photon counting data according to some embodiments of the present disclosure;

FIG. 4 is a block diagram illustrating an exemplary cascading of a plurality of sub-models according to some embodiments of the present disclosure;

FIG. 5 is a schematic diagram illustrating an exemplary pulse pileup effect according to some embodiments of the present disclosure;

FIG. 6 is a schematic diagram illustrating exemplary pulse waveforms under different pulse pileup orders according to some embodiments of the present disclosure;

FIG. 7A is a schematic diagram illustrating an exemplary content of a spectrum model according to some embodiments of the present disclosure;

FIG. 7B is a schematic diagram illustrating an exemplary process for calibrating a spectrum model according to some embodiments of the present disclosure;

FIG. 8 is a schematic diagram illustrating a changing trend in the highest pulse pileup order that contributes more than 1% % to a total contribution to an output signal under different total incident count rates according to some embodiments of the present disclosure;

FIG. 9 is a schematic diagram illustrating exemplary basis material decomposition according to some embodiments of the present disclosure;

FIG. 10 is a flowchart illustrating an exemplary process of image reconstruction according to some embodiments of the present disclosure;

FIG. 11 is a schematic diagram illustrating an exemplary process of image reconstruction according to some embodiments of the present disclosure;

FIG. 12A is a schematic diagram illustrating a complete energy spectrum of incident photons according to some embodiments of the present disclosure;

FIG. 12B is a schematic diagram illustrating an energy spectrum processed by an energy deposition sub-model according to some embodiments of the present disclosure;

FIG. 12C is a schematic diagram illustrating an energy spectrum processed by an energy resolution sub-model according to some embodiments of the present disclosure;

FIG. 12D is a schematic diagram illustrating an energy spectrum processed by a charge-sharing sub-model and a pulse pileup sub-model according to some embodiments of the present disclosure;

FIG. 12E is a schematic diagram illustrating an energy spectrum processed by a spectrum model according to some embodiments of the present disclosure;

FIG. 13A is a comparison plot illustrating calculated photon counting data corresponding to a low-energy bin versus validated test data;

FIG. 13B is a comparison plot illustrating calculated photon counting data corresponding to high-energy bin versus validated test data;

FIG. 13C is a comparison plot illustrating calculated photon counting data after merging a low-energy bin and a high-energy bin versus validated test data;

FIG. 14A is a schematic diagram illustrating a material line integral of AI of a target material line integral obtained by a process of image reconstruction according to some embodiments of the present disclosure;

FIG. 14B is a schematic diagram illustrating a material line integral of polymethyl methacrylate (PMMA) of a target material line integral obtained by a process of image reconstruction according to some embodiments of the present disclosure;

FIG. 14C is a schematic diagram illustrating a material line integral corresponding to a low-energy bin (LE) of a target material line integral obtained by a process of image reconstruction according to some embodiments of the present disclosure;

FIG. 14D is a schematic diagram illustrating a material line integral corresponding to a high-energy bin (HE) of a target material line integral obtained by a process of image reconstruction according to some embodiments of the present disclosure;

FIG. 14E is a schematic diagram illustrating a material line integral corresponding to a photon energy of 70 keV of a target material line integral obtained by a process of image reconstruction according to some embodiments of the present disclosure;

FIG. 15A is a schematic diagram illustrating a material line integral of AI of a material line integral obtained by a simple empirical model;

FIG. 15B is a schematic diagram illustrating a material line integral of PMMA of a material line integral obtained by a simple empirical model;

FIG. 15C is a schematic diagram illustrating a material line integral corresponding to a 50 mA tube current of a material line integral obtained by a simple empirical model;

FIG. 16 is a schematic diagram illustrating a reconstructed image obtained by reconstructing based on a target material line integral corresponding to a 150 mA tube current of a process of image reconstruction according to some embodiments of the present disclosure; and

FIG. 17 is a schematic diagram illustrating a reconstructed image obtained by reconstructing a material line integral corresponding to a 50 mA tube current obtained by a simple empirical model.

### DETAILED DESCRIPTION

In order to more clearly illustrate the technical solutions of the embodiments of the present disclosure, the following briefly introduces the drawings that need to be used in the description of the embodiments. Apparently, the accompanying drawings in the following description are only some examples or embodiments of the present disclosure, and those skilled in the art can also apply the present disclosure to other similar scenarios according to the drawings without creative efforts. Unless obviously obtained from the context or the context illustrates otherwise, the same numeral in the drawings refers to the same structure or operation.

It should be understood that “system”, “device”, “unit” and/or “module” as used herein is a method for distinguishing different components, elements, parts, portions or assemblies of different levels. However, the words may be replaced by other expressions if other words can achieve the same purpose.

As indicated in the disclosure and claims, the terms “a”, “an”, “an” and/or “the” are not specific to the singular form and may include the plural form unless the context clearly indicates an exception. Generally speaking, the terms “comprising” and “including” only suggest the inclusion of clearly identified steps and elements, and these steps and elements do not constitute an exclusive list, and the method or device may also contain other steps or elements.

The flowchart is used in the present disclosure to illustrate the operations performed by the system according to the embodiments of the present disclosure. It should be understood that the preceding or following operations are not necessarily performed in the exact order. Instead, various steps may be processed in reverse order or simultaneously. Meanwhile, other operations may be added to these procedures, or a certain step or steps may be removed from these procedures.

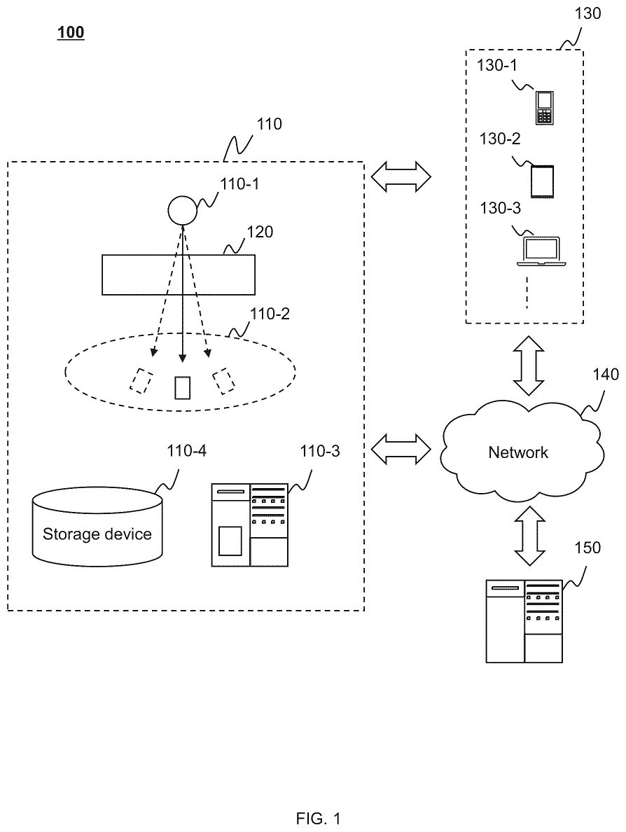

FIG. 1 is a schematic diagram illustrating an exemplary PCCT system PCCT system according to some embodiments of the present disclosure.

As shown in FIG. 1, the PCCT system 100 may include a scanning device 110, a terminal 130, a network 140, and a processing device 150.

The scanning device 110 may be configured to emit radiation rays to irradiate a subject 120 and generate a corresponding response to the radiation rays passing through the subject 120.

In some embodiments, the scanning device 110 may perform data or instruction acquisition, processing, and/or output, or the like. In some embodiments, the scanning device 110 may include a PCCT device. In some embodiments, the scanning device 110 may be a multi-modality scanning device, such as a PCCT-radiation therapy device, a PCCT-magnetic resonance imaging (MRI) device, etc.

The scanning device 110 may include one or more components. For example, the scanning device 110 may include a radiation source 110-1, a detecting device 110-2, an electronic component 110-3, a storage device 110-4, or the like, or a combination thereof. The radiation source 110-1 may be configured to emit radiation rays (e.g., X-rays, γ-rays, etc.) to irradiate the subject 120. The detecting device 110-2 may include a photon counting detector (PCD). The PCD may include a semiconductor material and a detector pixel unit. The detector pixel unit may include pixelated anodes. The detecting device 110-2 may include multiple electronic components 110-3 (e.g., a processor) each of which corresponds to one of the pixelated anodes. The multiple electronic components 110-3 (e.g., a processor) may be connected with the semiconductor material via multiple welding balls which may be used to make the PCD detector to be pixelated. Each of the multiple welding balls may correspond to a pixel. A pixelated anode, an electronic component, and a portion of the semiconductor material may constitute a detector pixel. An electronic component may include an amplifier, a threshold comparator, a counter, a readout circuit, etc. The PCD and the corresponding or included electronic component thereof may be configured to receive the radiation rays and/or process the received radiation rays, including data processing involved in an imaging process, such as receiving, counting, converting, and energy spectrum measurement. For example, the PCD and the corresponding or included electronic component thereof may receive the radiation rays after passing through the subject 120 and generate corresponding photon counting data (e.g., photon counting measurements, or spectrum measurements, or energy spectrum measurements). In some embodiments, the detecting device 110-2 may include a plurality of detecting modules arranged along the circumference direction of the scanning device 110. Each of the plurality of detecting modules may include multiple PCDs.

In some embodiments, the scanning device 110 may receive an instruction related to calibration and/or an instruction related to image reconstruction from the terminal 130 through a communication interface of the scanning device 110 or via the network 140. In some embodiments, the scanning device 110 may receive the instruction related to calibration and/or the instruction related to image reconstruction from a user through an interaction interface (not shown in FIG. 1) of the scanning device 110. In some embodiments, the scanning device 110 may obtain parameters related to scanning, parameters related to phantoms, or the like. The scanning device 110 may transmit the instructions, the parameters, and the data (e.g., intermediate data, a calibration result, or a reconstructed image) between the radiation source 110-1, the detecting device 110-2, the electronic unit 110-3, the storage device 110-4, and/or the terminal 130. In some embodiments, the electronic unit 110-3 and the storage device 110-4 may be located inside or outside of the scanning device 110, and may be in signaling connection with one or more components included in the scanning device 110. For example, the electronic unit 110-3 or a functional module capable of implementing functions of the electronic unit 110-3 may be integrated into the scanning device 110, or other possible system components.

The storage device 110-4 may include one or more storage components. Each storage component may be a standalone device or may be part of another device. In some embodiments, the storage device 110-4 may be configured to store the instructions, the parameters, and the data that may be involved in a CT imaging process. In some embodiments, the storage device 110-4 may include a random access memory (RAM), a read-only memory (ROM), a mass memory, a removable memory, a volatile read/write memory, or the like, or any combinations thereof.

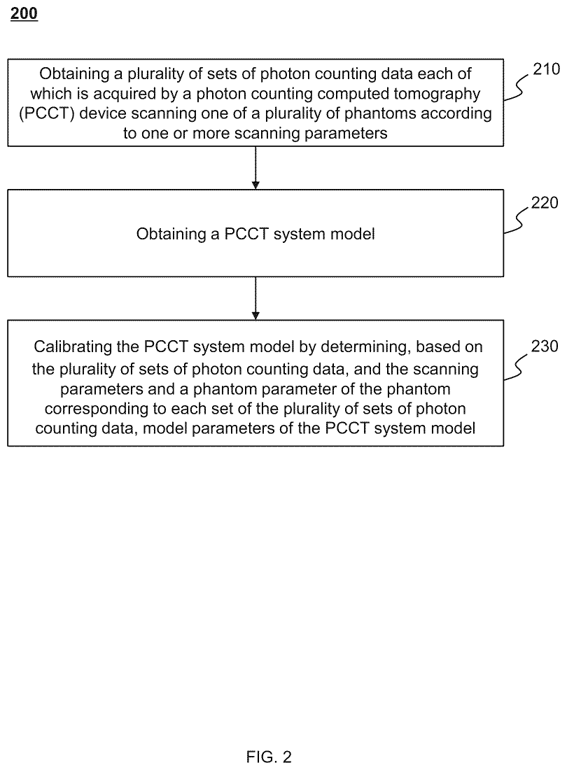

The subject 120 may be a phantom or a target subject (e.g., a human body, or an animal body). In some embodiments, the phantom may be used to analyze and/or adjust the imaging performance of the scanning device 110. More descriptions regarding the phantom may be found in FIG. 2 and related descriptions thereof.

The terminal 130 refers to one or more terminal devices or software used by the user. The terminal 130 may be configured to input the instruction related to calibration or the instruction related to image reconstruction and output the instruction related to calibration or the instruction related to image reconstruction to the scanning device 110 through the communication interface or via the network 140. The terminal 130 may also obtain a calibration result, an image reconstruction result, or the like. The terminal 130 may include a processing unit, a display unit, an input/output unit, a transmission unit, a storage unit, or the like. In some embodiments, the terminal 130 may be a mobile device 130-1, a tablet computer 130-2, a laptop computer 130-3, or any other device with input and/or output capabilities, or any combination thereof. The foregoing examples are intended only to illustrate the breadth of the range of the terminal 130 and are not intended to limit the scope of the terminal 130.

The network 140 facilitates the connection of components of the system and/or the connection of the system to external resource parts. The network 140 enables communication between components and with other parts outside the system to facilitate the exchange of data and/or information. In some embodiments, the network 140 may be one or more of a wired network or a wireless network. For example, the network 140 may include a cable network, a fiber optic network, a telecommunications network, the Internet, a local area network (LAN), a wide area network (WAN), a wireless local area network (WLAN), a metropolitan area network (MAN), a public switched telephone network (PSTN), a Bluetooth network, a ZigBee network (ZigBee), near field communication (NFC), an in-device bus, an in-device line, cable connection, or any combination thereof. The network connection between various parts may be made in one of the ways or may be made in multiple ways. In some embodiments, the network may be in various topologies such as point-to-point, shared, centralized, or a combination of topologies. In some embodiments, the network 140 may include one or more network access points.

The processing device 150 may process an instruction, one or more parameters (e.g., scanning parameters, model parameters, phantom parameters), the data (e.g., photon counting measurements), or the like, obtained from the components of the PCCT system 100. The processing device 150 may perform one or more of the functions described in the present disclosure by executing program instructions. In some embodiments, the processing device 150 may perform a calibration of a PCCT system model. For example, the processor 150 may control the radiation source 110-1 to scan each of a plurality of phantoms according to one or more scanning parameters and receive a plurality of sets of photon counting data from the detecting device 110-2. The processing device 150 may obtain the scanning parameters and one or more phantom parameters, e.g., an attenuation coefficient and thickness (which may be input by the user through the interaction interface of the scanning device 110, the terminal 130, etc., or generated by the processing device 150 after relevant data is received) of each of the plurality of phantoms. The processing device 150 may perform the calibration for a PCCT system model by determining model parameters of the PCCT system model based on the plurality of sets of photon counting data, the scanning parameters, and the phantom parameters, thereby obtaining a calibrated PCCT system model. In some embodiments, the processing device 150 may obtain one or more images of the subject 120 based on the calibrated PCCT system model. For example, the processing device 150 may control the scanning device 110 to scan the subject 120 and obtain photon counting measurements of the subject 120. The processing device 150 may determine the material parameters of one or more reference materials corresponding to the subject 120 based on the scanning parameter of the subject 120, the photon counting measurements of the subject 120, and the calibrated PCCT system model. The processing device 150 may determine one or more images of the subject 120 based on the material parameters of the one or more reference materials corresponding to the subject 120. In some embodiments, the processing device 150 may include one or more sub-processing devices (e.g., a single-core processing device or a multi-core processing device). Merely by way of example, the processing device 150 may include a central processing unit (CPU), an application-specific integrated circuit (ASIC), an application-specific instruction processor (ASIP), a graphics processor (GPU), a physical processor (PPU), a digital signal processor (DSP), a field-programmable gate array (FPGA), a programmable logic circuit (PLD), a controller, a microcontroller unit, reduced instruction set computer (RISC), microprocessor, or the like, or any combination thereof.

It should be noted that the description of the system and its modules shown in the application scenario 100 is for ease of descriptive illustration only and does not limit the present disclosure to the scope of the embodiments. It may be understood that for those skilled in the art, after understanding the principle of the system, it may be possible to arbitrarily combine the individual modules or form a subsystem to be connected with other modules without departing from the principle. In some embodiments, the radiation source 110-1, the detecting device 110-2, the electronic unit 110-3, the storage device 110-4, or the like, disclosed in FIG. 1, may be different modules in a single system, or one module realizing the functions of two or more of the above modules. For example, multiple detectors may be arranged in the PCCT system 100 and each detector may share one electronic unit, and each detector may also have its electronic unit. Such variations are within the scope of protection of the present disclosure.

FIG. 2 is a flowchart illustrating an exemplary process for calibrating a PCCT system model according to some embodiments of the present disclosure. In some embodiments, the calibration process for the PCCT system model may be performed by the electronic component 110-3 or the processing device 150. For example, process 200 may be stored in a storage device (e.g., the storage device 110-4) in the form of a program or instruction, and when the processing device 150 executes the program or instruction, process 200 may be implemented. The schematic diagram illustrating the operation of process 200 below is illustrative. In some embodiments, the process may be completed by one or more additional operations not described and/or one or more operations not discussed. As shown in FIG. 2, process 200 for calibrating the PCCT system model may include the following operations. In addition, the order of the operations of the process 200 illustrated in FIG. 2 and described below is non-limiting.

As used herein, the calibration of the PCCT system model refers to determining/obtaining one or more model parameters of the PCCT system model. The determination of a model parameter may include determining a target value of the model parameter. A target value of a model parameter may be denoted as a vector, a matrix, an image, etc. In some embodiments, the PCCT system model may be in the form of a function. The model parameters of the PCCT system model may serve as constants of the function, scanning parameters and reference material parameters of the PCCT system model may serve as independent variables of the function, and photon counting data may serve as a dependent variable of the function. Thus, the calibrated PCCT system model may provide a mapping relationship between a scanning parameter, a reference material parameter, and photon counting data through/based on the determined model parameters.

In some embodiments, the calibration of the PCCT system model may be implemented on each detector pixel of a PCCT device. In other words, each detector pixel in the detecting device may correspond to one or more model parameters of the PCCT system model. The calibration of the PCCT system model implemented on a detector pixel may be performed based on photon counting data acquired by the detector pixel according to operations 210-230.

In 210, a plurality of sets of photon counting data may be obtained. Each of the plurality of sets of photon counting data may be acquired by a photon counting computed tomography (PCCT) device scanning one of a plurality of phantoms according to one or more scanning parameters.

In some embodiments, each set of the plurality of sets of photon counting data may be acquired by a photon counting computed tomography (PCCT) device scanning one of a plurality of phantoms according to one or more scanning parameters. The set of photon counting data acquired by the PCCT device scanning a phantom may also referred to as a set of photon counting measurements or spectral measurements. In some embodiments, at least one set of the plurality of sets of photon counting data may be acquired according to a simulation algorithm, e.g., a Monte Carlo algorithm. The set of photon counting data acquired via a simulation algorithm may also referred to as photon counting simulation or spectral simulation.

In some embodiments, at least two of the plurality of phantoms may have different phantom parameters. A phantom parameter of a phantom may include the thickness of the phantom, the type of the phantom material, a material line integral of the phantom, an attenuation coefficient (e.g., an attenuation coefficient curve or function) of the phantom, etc. As described herein, the thickness of a phantom refers to the thickness of each of one or more materials included in the phantom; the attenuation coefficient of a phantom refers to the attenuation coefficient of each of one or more materials included in the phantom; and the material line integral of the phantom refers to a material line integral of each of one or more materials included in the phantom. The thickness of the material of a phantom refers to a length in the direction of a minimum path of radiation rays passing through the material of the phantom. Two phantoms having different phantom parameters refer to that at least one phantom parameter in the same type of the two phantoms is different. For example, if the materials of two phantoms are different, the two phantoms may have different phantom parameters. As another example, if the thicknesses of two phantoms are different, the two phantoms may have different phantom parameters.

In some embodiments, each of the plurality of phantoms may include one single material, such as PMMA, Al, etc. In some embodiments, each of the plurality of phantoms may include one single material and two different phantoms among the plurality of phantoms may include different materials. For example, each of the plurality of phantoms may correspond to one material. In some embodiments, the plurality of phantoms with different materials may have the same thickness. In some embodiments, the plurality of phantoms with different materials may have different thicknesses. In some embodiments, the plurality of phantoms may have the same material. The plurality of phantoms with the same material may have different thicknesses.

In some embodiments, each of the plurality of phantoms may include a plurality of materials, such as a combination of PMMA and aluminum. In some embodiments, the materials included in the plurality of phantoms may be the same. In some embodiments, the plurality of phantoms with the same materials may have different thicknesses. In some embodiments, the materials included in the plurality of phantoms may be different.

In some embodiments, each of a portion of the plurality of phantoms may include one single material and each of another portion of the plurality of phantoms may include different materials.

In some embodiments, a phantom may include a plate. The plurality of phantoms may include a series of plates with different thicknesses. The material of each of the series of plates may include, for example, PMMA and/or AI (the combination material of PMMA and Al). In some embodiments, the size of a phantom may be the same as or similar to the radiation range of radiation rays generated and emiited by the PCCT device, so that more radiation rays emitted by the radiation source pf a PCCT device may pass through a phantom located within the radiation range of the PCCT device and be received by a detector to produce relatively complete photon counting measurements, thereby minimizing data loss. In some embodiments, the length and the width of the phantom may cover an entire collimated X-ray field (i.e., the axial field of view (FOV)). In other words, the length of the phantom along the first axial of the PCCT device (i.e., an axial parallel to a direction along which a scanning table enters the radiation range of the PCCT device) may be the same as or similar to the length of the collimated X-ray field along the first axial. The width of the phantom along the second axial of the PCCT device (i.e., an axial perpendicular to the first axial and located at the same plane with the first axial) may be the same as or similar to the width of the collimated X-ray field along the second axial. When the plurality of phantoms are scanned, each of the plurality of phantoms may be placed in the radiation range (i.e., a scanning region). During a scanning process, each of the plurality of phantoms may be in a stationary state without rotating. In some embodiments, during the scanning process, the radiation source and the detector of the PCCT device may be in a stationary state without rotating.

In some embodiments, the plurality of phantoms may be integrated on a support. For example, the support may be a cylindrical structure. The cylindrical structure may include a plurality of holes for placing different phantoms. The different phantoms may have the same material or different materials. In some embodiments, the different phantoms with the same material may have different thicknesses. In some embodiments, the different phantoms with different materials may have the same thickness. When the plurality of phantoms is scanned, the support with the plurality of phantoms may be placed on a rotation shaft of a rotary gantry. The plurality of phantoms may be rotated by controlling the rotation shaft, thereby scanning the plurality of phantoms from different angles.

The plurality of phantoms may be preset. Phantom parameters related to the plurality of phantoms may be determined by an operator. The processor may obtain the phantom parameters input by the operator through a communication interface or an interaction interface, or retrieve pre-stored phantom parameters from the storage device.

A phantom may correspond to a thickness, an attenuation coefficient, and/or a material line integral. The material line integral refers to an integral of an attenuation coefficient in the transmission path of a radiation ray, which reflects an attenuation of the radiation ray of a certain energy in the phantom when the radiation ray passes through the phantom. The material line integral of a phantom may be related to the thickness of each of the one or more materials of the phantom and the types of the one or more materials of the phantom. The types of the one or more materials of the phantom may determine the attenuation coefficient. Different thicknesses and/or different material types may correspond to different material line integrals. The material line integral of a phantom may be determined based on the thickness of the phantom and the type of material of the phantom. For example, material line integral of the phantom may be obtained through at least one of theoretical calculation, experimental measurement, numerical simulation, and reference databases.

In some embodiments, each of the plurality of phantoms may correspond to a combination of an attenuation coefficient and a material thickness. For example, for a set of phantoms, each phantom of the set of phantoms may be made of at least one of two or more materials, and different phantoms may include materials with different thicknesses. For example, for a phantom 1, . . . , a phantom M, wherein M is an integer of 2, 3, 8, etc., each of the M phantoms may include a certain thickness of PMMA and/or a certain thickness of Al. The thickness of PMMA in phantom 1 may be different from the thickness of PMMA in other phantoms (e.g., the phantom M). As a further example, phantom 1 may consist of PMMA with a thickness of I₁ and AI with a thickness of I₂, an attenuation coefficient corresponding to phantom 1 may include an attenuation coefficient μ₁ corresponding to PMMA, and an attenuation coefficient μ₂ corresponding to AL, and a material thickness corresponding to the phantom 1 may be “I₁, I₂”. The total thickness of the phantom 1 may be “I₁+I₂”.

In some embodiments, the material line integral corresponding to a material of a phantom may be determined based on an attenuation coefficient of the material and the material thickness of the material. For example, the attenuation coefficient of the material may be obtained from a storage device, and the material line integral corresponding to the material of the phantom may be determined based on the material thickness and the attenuation coefficient of the phantom according to the Beer-Lambert Law. In some embodiments, the phantom may include a first material and a second material. The first material may have a first thickness and a first attenuation coefficient. The second material may have a second thickness and a second attenuation coefficient. The material line integral corresponding to the phantom may include a first material line integral corresponding to the first material and a second material line integral corresponding to the second material. In some embodiments, the material line integral corresponding to the phantom may include a combination of the first material line integral corresponding to the first material and the second material line integral corresponding to the second material. For example, a first matrix may include first elements denoting the attenuation coefficient of the first material and the attenuation coefficient of the second material. A second matrix may include second elements denoting the thickness of the first material and the second material. The material line integral corresponding to the phantom may be a third matrix obtained by multiplying the first matrix and the second matrix. The material line integral corresponding to the phantom may be decomposed to generate the first matrix (i.e., the first material line integral corresponding to the first material) and the second matrix (i.e., the second material line integral corresponding to the first material).

A scanning parameter corresponding to a phantom refers to a parameter relating to an operation of the PCCT device for scanning the phantom. The scanning parameter may include a scanning range, a scanning time, a tube current, a tube voltage, or the like, or a combination thereof. In some embodiments, a user may set the scanning parameters corresponding to different phantoms, and store the scanning parameters in the storage device, or directly send the scanning parameters to the scanning device (e.g., the PCCT device). The scanning device may acquire the scanning parameters and scan the phantom based on the scanning parameters.

In some embodiments, the scanning parameters in the same type corresponding to different phantoms may be different. For example, the same phantom may be scanned at a first tube current and a second tube current, respectively. The first tube current may be greater than the second tube current. The first tube current is also referred to as a high tube current, such as 100 mA, 150 mA, 200 mA, etc. The second tube current is also referred to as a low tube current, such as 10 mA, 20 mA, 50 mA, etc. The first tube currents corresponding to at least two phantoms may be different, and the second tube currents corresponding to the at least two phantoms may be different. As another example, the first tube currents corresponding to the at least two phantoms may be the same, and the second tube currents corresponding to the at least two phantoms may be different. As still another example, the first tube currents corresponding to the at least two phantoms may be different, and the second tube currents corresponding to the at least two phantoms may be the same. It should be noted that the first tube current and the second tube current are merely provided for illustration, and a phantom may be scanned under more than two different tube currents.

When the phantom is scanned, a radiation exposure of the radiation source of the scanning device may be determined according to the scanning parameters. The radiation source may be controlled to emit radiation rays to the phantom from at least one angle around the phantom, thereby realizing scanning of the phantom.

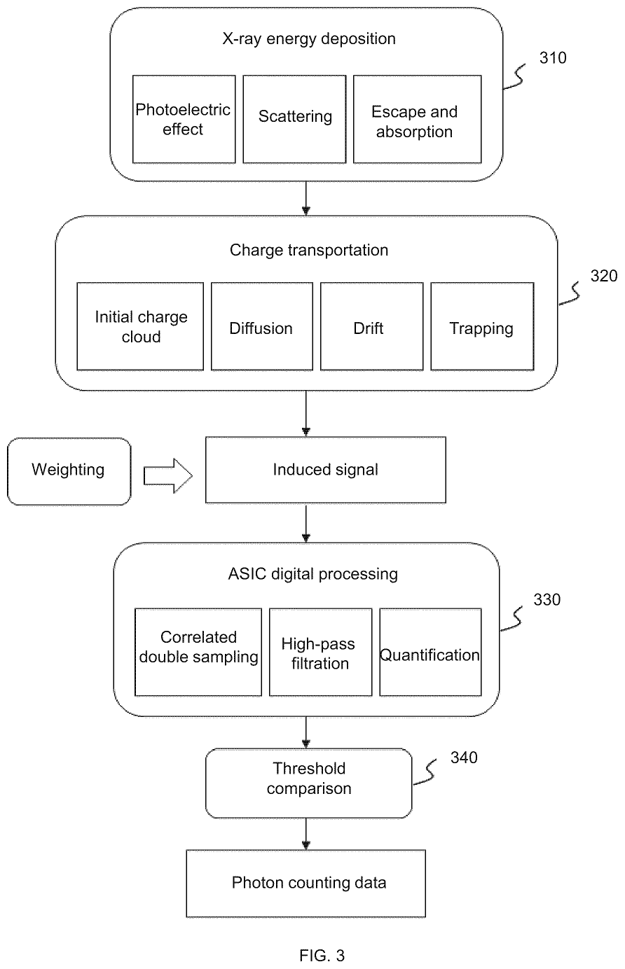

In some embodiments, the scanning parameter may include at least one tube current. In some embodiments, the plurality of sets of photon counting data may be obtained by performing multiple times of scans. For each time of scan, a set of photon counting data may be obtained by scanning a phantom based on at least one tube current (e.g., the first tube current and the second tube current). More descriptions regarding generating and obtaining the photon counting measurement may be found in FIG. 3 and related descriptions thereof.

In some embodiments, the plurality of sets of photon counting data may be obtained based on different scanning parameters. For example, the different scanning parameters may include a plurality of tube currents, such as 20 mA, 50 mA, 100 mA, 150 mA, and/or 200 mA, etc. As a further example, the plurality of sets of photon counting data may include a set of photon counting measurements 1, a set of photon counting measurements 2, . . . , a set of photon counting measurements M. The set of photon counting measurements 1, the set of photon counting measurements 2, . . . , the set of photon counting measurement M may be obtained, respectively, by scanning the phantom 1, the phantom 2, . . . , the phantom M based on one or more corresponding tube currents.

In some embodiments of the present disclosure, the plurality of sets of photon counting data may be obtained by scanning the plurality of phantoms, so that as much data as possible for calibration can be obtained, potential errors caused by data noise and chance events can be reduced, and the accuracy of the calibration result can also be improved. Furthermore, the calibration results can comprehensively cover various scanning parameters, the plurality of phantoms, or target subjects, thereby expanding the range of applications.

In some embodiments, a set of photon counting data may include multiple subsets of photon counting data. Each of the multiple subsets of photon counting data may correspond to an energy interval (i.e., an energy bin). A subset of photon counting data may also be referred to as a photon counting subset. A set of photon counting data may also be referred to as a photon counting set.

In some embodiments, for each of the plurality of phantoms, after the radiation source emits radiation rays to irradiate the phantom at one or more tube currents, the radiation rays passing through the phantom may interact with a material of the detector to generate electron-hole pairs. An electron may drift to the anode of the detector and an electrical signal (e.g., a voltage pulse) may be generated. The amplitude of the electrical signal may be proportional to the energy of radiation rays received by the detector for generating the electrical signal. The amplitude of the electrical signal after a series of processing (e.g., filtration, quantization, etc.) may be compared with different energy thresholds. If the amplitude of the electrical signal exceeds an energy threshold, the electrical signal may be counted. If the amplitude of the electrical signal is located within an energy interval defined by two energy thresholds, the electrical signal may be counted to belong to an energy bin or energy interval defined by the two energy thresholds. Then the photon counting data (i.e., the photon counting subset) corresponding to each energy interval or energy bin may be obtained.

In some embodiments, for each of the plurality of phantoms, the radiation source may scan the phantom at a plurality of angles at one tube current, and then the set of photon counting data generated by the detector may include photon counting subsets each of which corresponds to one energy interval at the plurality of angles.

In 220, a PCCT system model may be obtained.

The PCCT system model may represent the response features of a PCCT system. For example, when the radiation rays are projected onto a detector after passing through a phantom or a target subject (e.g., the patient), the detector may generate electrical signals, and the PCCT system model may describe a relationship between the electrical signals generated by the detector and radiation rays emitted by the radiation sources. The PCCT system model may also be referred to as a PCD measurement model.

The PCCT system model may be a previously calibrated PCCT system model or a PCCT system model that has never been calibrated. For example, the PCCT system model may be calibrated periodically or aperiodically. The PCCT system model obtained in operation 220 may be a calibrated PCCT system model that was calibrated last time. The previously calibrated PCCT system model may be updated according to operation 240.

In some embodiments, the PCCT system model may be a physical model based on a photon transmission path. The physical model based on the photon transmission path may consider an X-ray source intensity and spectrum, imaging object (e.g., the plurality of phantoms or other target subject) filtration, collimation filtration, X-ray absorption in the semiconductor, charge cloud formation and collection, pixel readout (including charge sharing and pulse pileup effects), energy binning, and other physical effects to map the incident photons to spectrum information, thereby realizing measurement and analysis of X-ray energy distribution.

In some embodiments, the PCCT system model may include a first portion. The first portion may be related to the generation and the emission of the radiation rays (e.g., X-rays). In some embodiments, the first portion may include a first sub-model. The first sub-model may be configured to characterize a relationship between a source spectrum and the scanning parameters, such as a relationship between the source spectrum and the tube voltage and/or the tube current, etc. The source spectrum refers to the initial energy spectrum generated and emitted by the radiation source of the scanning device.

In some embodiments, the PCCT system model may include a second portion. The second portion may be related to a process of radiation absorption (or attenuation or filtration). In some embodiments, the second portion may include a second sub-model. The second sub-model may be configured to characterize a relationship between the transmissivity of the radiation rays in an incident object (e.g., a collimator, a subject to be scanned) and one or more parameters (e.g., energy) of the radiation rays, one or more parameters of the incident object, etc. The incident object may include one or more collimators located between the subject to be scanned and the radiation source, the subject to be scanned (e.g., a phantom, a human body, etc.). The parameters of the incident object may include a mass attenuation factor (i.e., an attenuation coefficient), the thickness, the density, etc., of the incident object, compositions of the incident object, etc.

In some embodiments, different second sub-models may be provided for different types of incident objects. For example, when the incident object is a collimator, the second sub-model may be configured to characterize a relationship between the transmissivity of the radiation rays in the collimator and the X-ray parameters (e.g., energy), the parameters of the collimator, etc. As another example, when the incident object is a subject to be scanned (e.g., a phantom), the second sub-model may be configured to characterize a relationship between the transmissivity of the radiation rays in the subject to be scanned and the X-ray parameters (e.g., energy), the parameters of the subject to be scanned, etc.

In some embodiments, the PCCT system model may include a third portion. The third portion may be related to the response of the detector. In some embodiments, the third portion may include a third sub-model. The third sub-model may be configured to characterize the response of the detector in at least one process of X-ray generation, X-ray absorption, energy deposition, energy resolution, charge sharing, pulse pileup effect, and energy binning in the generation process of photon counting data.

In some embodiments, the PCCT system model may be formed by a plurality of submodels through cascading. As described herein, cascading of the plurality of submodels means that the plurality of submodels is connected by multiplication or convolution.

In some embodiments, the PCCT system model may include one or more spectrum models each of which corresponds to an energy bin. In some embodiments, the PCCT system model may be a sum of one or more spectral models corresponding to one or more energy bins. More descriptions for the PCCT system model may be found elsewhere in the present disclosure (e.g., FIGS. 3-4 and 7A and 7B, and the related descriptions thereof).

In some embodiments, photon counting data of a subject (e.g., the phantom or a target subject) generated by the detector may be affected by at least one of the material thickness of the subject, the attenuation coefficient of the subject, the scanning parameters of the subject, an incident count rate, or the like, or a combination thereof. In some embodiments, the PCCT system model may describe a relationship between the photon counting data, at least one of the material thickness, the attenuation coefficient, the scanning parameters, the incident count rate, or the like, etc.

The PCCT system model to be calibrated may include model parameters to be determined or updated. As described herein, the model parameters to be determined or updated refer to values of the model parameters to be determined or updated. The accuracy of the determined model parameters may affect the accuracy of the relationship between the electrical signals generated by the detector described in the PCCT system model and the incident photons.

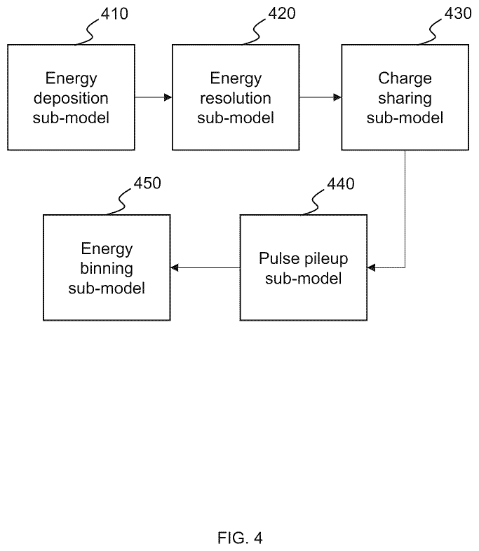

In some embodiments, the model parameters may be related to at least one of X-ray generation, X-ray filtration, X-ray absorption, energy deposition, energy resolution, charge sharing, pulse pileup effect, and energy binning in the generation process of the photon counting data. More descriptions regarding X-ray generation, X-ray filtration, X-ray absorption, energy deposition, energy resolution, charge sharing, pulse pileup effect, and energy binning may be found in FIG. 4 and related descriptions thereof.

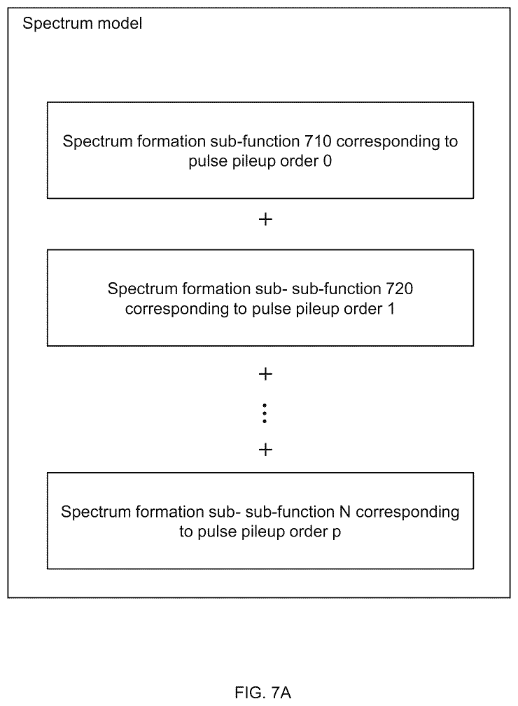

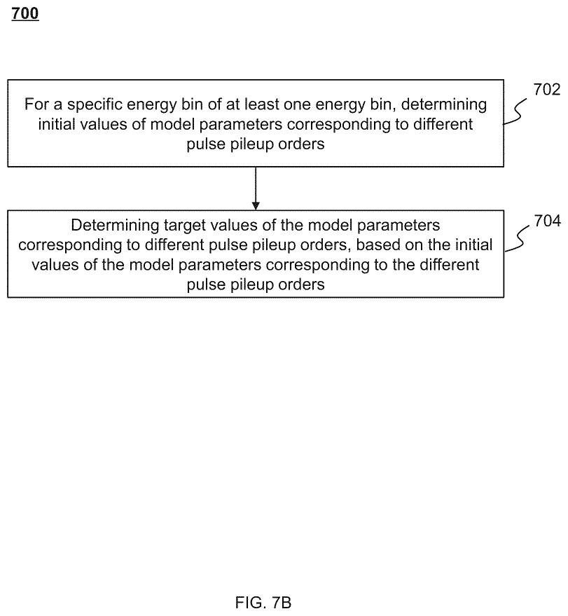

In some embodiments, the model parameters may include one or more spectrum generation parameters. A spectrum generation parameter may be related to the physical effect generated during the generation process of the photon counting data. For example, the physical effect may be generated in X-ray generation, X-ray filtration, X-ray absorption, energy deposition, energy resolution, charge sharing, pulse pileup effect, and energy binning in the generation process of the photon counting data. In some embodiments, the spectrum generation parameters may affect the shape of an energy spectrum. The PCCT system model including the spectrum generation parameters may reflect an energy distribution of the incident photons. For example, the spectrum generation parameters may include influence coefficients of energy response spectra at a plurality of different pulse pileup orders, etc. More descriptions regarding the energy response spectra, the pulse pileup orders, etc., may be found in FIG. 7A and related descriptions thereof.

In some implementations, the model parameters may include spectrum influence parameters. The spectrum influence parameters may be related to the data process of the detector (e.g., data process by an electronic component of the detector) after radiation rays are detected by the detector. For example, the spectrum influence parameters may include a performance parameter of the detector, such as the dead time, the incident count rate, the detection efficiency, etc. More descriptions regarding the dead time and the incident count rate may be found in FIG. 7A and FIG. 7B and related descriptions thereof.

In 230, the PCCT system model may be calibrated by determining, based on the plurality of sets of photon counting data, and the scanning parameters and phantom parameters of the phantom corresponding to each set of the plurality of sets of photon counting data, model parameters of the PCCT system model.

In some embodiments, the calibrated PCCT system model may represent a mapping relationship between photon counting data (reference photon counting data), one or more scanning parameters (reference scanning parameters), and one or more material parameters of one or more reference materials (also referred to as reference material parameters) via the determined model parameters.

In some embodiments, the calibrated PCCT system model may be in the form of a function. The one or more scanning parameters and the one or more reference material parameters may be independent variables of the function. The photon counting data may be a dependent variable of the function. The determined model parameters may be constants of the function. Any object may be considered as a mixture of two or more different base substances (i.e., base materials). An attenuation coefficient of each voxel within the object may be represented based on a combination of attenuation coefficients of different base materials. A base material may also be referred to as a reference material. The scanning parameters may include a tube current, a tube voltage, etc. The reference material parameters may include attenuation coefficients of the one or more reference materials and thicknesses of the one or more reference materials. In some embodiments, the types of the reference materials may be a default setting of the system or set by an operator of the system 100. If the reference materials are determined, the attenuation coefficients of the reference materials may be determined. The attenuation coefficients of the reference materials may be the constants of the function. The independent variables of the function may include the thicknesses of the one or more reference materials and the one or more scanning parameters. In some embodiments, the reference material parameters may include a factor. The attenuation coefficient of another reference material may be denoted by the factor and the attenuation coefficient of a reference material set by the operator.

In some embodiments, the processor may determine the model parameters of the PCCT system model by performing parameter fitting through a fitting algorithm, such as a least squares algorithm, a maximum likelihood estimation algorithm, a Gauss-Newton iteration algorithm, etc., based on the plurality of sets of photon counting data, and the scanning parameters and the phantom parameters corresponding to each set of the plurality of sets of photon counting data. More descriptions regarding the parameter fitting may be found in FIG. 7B and related descriptions thereof.

After the model parameters are determined, the calibrated PCCT system model may be obtained by substituting the determined model parameters into the PCCT system model.

The parameter fitting may be performed on the PCCT system model based on the plurality of sets of photon counting data and the scanning parameters and the phantom parameters corresponding to each set of the plurality of sets of photon counting data, and the calibrated PCCT system model may establish a correlation between the photon counting data, the phantom parameters (or a parameter of a reference material, i.e., a reference material parameter), and/or the scanning parameters via the determined model parameters.

In some embodiments, one or more specific reference material parameters (e.g., the thicknesses of the reference materials) may be determined through the calibrated PCCT system model based on specific photon counting data and specific scanning parameters when the types of the reference materials are specified. Furthermore, the material line integrals of the reference materials may be determined based on the determined thicknesses of the reference materials and attenuation coefficients corresponding to the specified reference materials. In some embodiments, a total material line integral may be determined based on specific photon counting data and specific scanning parameters. The total material line integral may be decomposed into at least two material line integrals of at least two reference materials when the types of the at least two reference materials are specified. The at least two material line integrals of at least two reference materials may be used to reconstruct two material decomposition images, respectively according to an image reconstruction algorithm, e.g., a filtered back-projection (FBP) algorithm.

In some embodiments, specific photon counting data may be determined through the calibrated PCCT system model based on specific reference material parameters (e.g., the thicknesses of the reference materials and the types of reference materials) and specific scanning parameters.

In some embodiments, one or more specific scanning parameters may be determined through the calibrated PCCT system model based on one or more specific reference material parameters (e.g., the thicknesses of the reference materials, and the types of the reference materials) and specific photon counting data.

A material line integral may be represented by the thickness of a material and the attenuation coefficient of the material. In some embodiments, the calibrated PCCT system model may represent a mapping relationship between the photon counting data, the one or more scanning parameters, and the material line integral. The material line integral may be determined through the calibrated PCCT system model based on specific photon counting data and one or more specific scanning parameters. In some embodiments, the specific photon count data may be determined when the material line integral and the scanning parameters are specified, or specific scanning parameters may be determined when the material line integral and the photon count data are specified.

In some embodiments of the present disclosure, the PCCT system model may be calibrated by performing parameter fitting on the PCCT system model to be calibrated based on the plurality of sets of photon counting data and the scanning parameters and the phantom parameters corresponding to the plurality of phantoms, so that the PCCT system model comprehensively considering the intensity and spectrum of the X-ray source, X-ray filtration (e.g., imaging object filtration and/or collimator filtration), and detector response features may be obtained. The intensity and spectrum of the X-ray source may be reflected via the scanning parameters. The imaging object filtration may be reflected by the phantom parameters (e.g., the thicknesses of the phantom materials, and the attenuation coefficients of the phantom materials) corresponding to the plurality of phantoms. The detector response features may be reflected by the photon counting data corresponding to the scanning parameters, the phantom parameters, and the model parameters. The resulting PCCT system model may be the physical model based on the photon transmission path, which has a more accurate energy response to the incident photons, and has a higher fitting degree to the actual data with a smaller error, thereby further accurately describing the energy response of the incident photons, being used for subsequent image reconstruction of the target subject, and being conducive to generating more accurate reconstructed images.

In some embodiments, the model parameters may include one or more groups. Each group of the one or more groups of model parameters may correspond to different pulse pileup orders under an energy bin. In other words, different energy bins may correspond to different model parameters. The count of the one or more groups may be the same as the count of the one or more energy bins. In some embodiments, each group of the one or more groups of model parameters may be determined based on photon counting data, in each set of the plurality of sets of photon counting data, that belong to the energy bin according to a fitting algorithm. For example, for each set of the plurality of sets of photon counting data, the set of photon counting data may be divided into multiple subsets under different energy bins. Each subset of the multiple subsets of photon counting data may also be referred to as a photon counting subset. A group of model parameters corresponding to an energy bin may be determined based on a photon counting subset in each set of the plurality of sets of photon counting data that belongs to the energy bin.

In some embodiments, the PCCT system model (or the calibrated PCCT system model) may include one or more spectrum models. Each of the one or more spectrum models may correspond to an energy bin of one or more energy bins. Each of the one or more spectrum models may represent a mapping relationship between photon counting data under the energy bin, the one or more scanning parameters, and the one or more parameters of the reference materials through/with the group of model parameters corresponding to the energy bin. More descriptions for the calibration of a spectrum model may be found elsewhere in the present disclosure (e.g., FIG. 7B and the descriptions thereof).

In some embodiments, the one or more spectrum models may be independent from each other. The calibration of the one or more spectrum models may be independent. In other words, the calibration of the one or more spectrum models corresponding to one or more energy bins may be performed separately according to the corresponding photon counting subsets. More descriptions for the calibration of a spectrum model may be found elsewhere in the present disclosure (e.g., FIG. 7B and the descriptions thereof).

In some embodiments, the one or more spectrum models may be integrated into one single model, i.e., the PCCT system model. For example, the PCCT system model may be a sum of the one or more spectrum models. The one or more spectrum models may be jointly calibrated. For example, initial values of each group of the one or more groups of model parameters may be determined. An iteration process including a plurality of iterations may be performed based on the initial values of each group of the one or more groups of model parameters. In the first iteration, a total photon counting estimation corresponding to a set of photon counting data among the plurality of sets of photon counting data may be determined based on the initial values of the each group of the one or more groups of model parameters. More descriptions for the initial values of the each group of the one or more groups of model parameters may be found elsewhere in the present disclosure. In some embodiments, the initial values may be the same as the target values of the model parameters determined in a previous calibration of the PCCT system model. The total photon counting estimation corresponding to the set of photon counting data may be a sum of photon counting estimations each of which corresponds to a photon counting subset that belongs to or corresponds to an energy bin. A photon counting estimation corresponding to an energy bin may be determined based on the initial values of one group of spectrum parameters and a spectrum model corresponding to the energy bin. The spectrum model corresponding to the energy bin may be denoted by the initial values of the group of model parameters corresponding to the energy bin. The photon counting estimation corresponding to the energy bin may be determined by inputting a photon counting subset in the set of photon counting data under the energy bin and the scanning parameters and phantom parameters corresponding to the set of photon counting data into the spectrum model with the initial values of the one group of model parameters. A target optimization function may be constructed based on an error between each set of the plurality of sets of photon counting data and the total photon counting estimation corresponding to each set of the plurality of sets of photon counting data. Whether a termination condition is satisfied may be determined. In response to determining that the termination condition is satisfied, the initial values of each group of the one or more groups of model parameters may be designated as target values of the each group of the one or more groups of model parameters. In response to determining that the termination condition is not satisfied, the initial values of the each group of the one or more groups of model parameters may be updated based on the value of target optimization function. The updated values of the each group of the one or more groups of model parameters may be designated as initial values of the each group of the one or more groups of model parameters in the next iteration (e.g., the second iteration). In the next iteration, another set of photon counting data may be used to update the initial values of each group of the one or more groups of model parameters. Other iterations may be performed similarly to the first iteration. The termination condition may include that the value of the target optimization function is converged, a count of iterations is performed, the value of the target optimization function is minimum, etc.

In some embodiments, the target optimization function may include a first item and a second item. The first item may be configured to constrain an error between each set of the plurality of sets of photon counting data and the total photon counting estimation corresponding to each set of the plurality of sets of photon counting data. The second item may include a regular term configured to constrain an error between projection data corresponding to the each set of the plurality of sets of photon counting data and projection data corresponding to the total photon counting estimation corresponding to each set of the plurality of sets of photon counting data in the projection domain.

In some embodiments, different scanning devices may correspond to different target values of the model parameters.

In some embodiments, different collimators may correspond to different target values of the model parameters. If the one or more collimators in the scanning device are changed, the calibrated PCCT system model may need to be calibrated or updated again.

In some embodiments, different tube voltages may correspond to different target values of the model parameters. If the tube voltage in the scanning device is changed, the calibrated PCCT system model may need to be calibrated or updated again. In some embodiments, the calibrated PCCT system model may need to be calibrated or updated again based on photon counting data acquired by the scanning device according to the changed tube voltage.

In some embodiments, different scanning devices, different collimators, and/or different tube voltages may correspond to the same target values of one or more groups of model parameters. The model parameters of the PCCT system may be determined based on photon counting data acquired under different scanning devices, different collimators, and/or different tube voltages.

It should be noted that the foregoing description of the calibration process for the PCCT system model is for the purpose of example and illustration only, and does not limit the scope of application of the present disclosure. For those skilled in the art, various corrections and changes may be made to the processes related to the calibration process for the PCCT system model under the guidance of the present disclosure. However, these corrections and changes remain within the scope of the present disclosure.

FIG. 3 is a schematic diagram illustrating an exemplary process of generating photon counting data according to some embodiments of the present disclosure. As shown in FIG. 3, the exemplary process of generating the photon counting data may include the following operations.

In 310, X-ray energy deposition may be performed. The X-ray energy deposition may include a photoelectric process, a scattering process, an escape and absorption process, etc. X-rays may interact with materials of the PCD to cause ionization or excitation, thereby generating charges in a free state.

In 320, charge transportation may be performed. The charge transportation may include a process of generating an initial charge cloud, a diffusion process, a drift process, a trapping process, etc. An induced signal may be generated through the charge transportation. A weighting processing may be performed on the induced signal based on a charge distribution situation in a semiconductor material, and a weighted signal may be input into an application specific integrated circuit (ASIC) for further digital processing.

In 330, the digital processing of the ASIC may be performed. The digital processing may include a correlated double sampling process, a high-pass filtration process, a quantification process, etc.

In 340, threshold comparison may be performed. The threshold comparison may be performed on the signal output from the operation 330. That is, the signal output from the operation 330 may be compared with a preset energy binning threshold, to obtain the photon counting data (counts).

FIG. 4 is a block diagram illustrating an exemplary cascading of a plurality of sub-models according to some embodiments of the present disclosure.

In some embodiments, the plurality of sub-models may include at least one of an energy deposition sub-model 410, an energy resolution sub-model 420, a charge sharing sub-model 430, a pulse pileup sub-model 440, and an energy binning sub-model 450.

The energy deposition sub-model 410 refers to a model based on a mechanism by which energy deposition occurs. In some embodiments, the processor may predict energy conversion of the photons in materials of the detector, and establish the energy deposition sub-model 410 based on the energy conversion. The energy deposition sub-model 410 may be characterized as a response function of the PCCT system during the process of energy deposition. For example, the energy deposition sub-model 410 may be constructed according to formula (1) and formula (2):

$${S}_{abs} ( i , j ) = {S}_{abs} ( {E}_{i} , {E}_{j} ) , \qquad ( 1 )$$

$${S}_{abs} ( {E}^{′} , E ) = {\sum }_{i} {p}_{i} ( {E}^{′} = Δ {E}_{i} ) , \qquad ( 2 )$$

wherein S_{abs} denotes the response function (which may be denoted as a matrix) for describing an energy deposition effect, i, j denotes elements in an ith row and a jth column of the matrix, i represents a serial number of a route in which incident photons are absorbed, and i may be an integer from 1 to 7; j represents the incident photon, and j may be an integer; S_{abs}(i, j) denotes a return value of the function S_{abs} with Eᵢ and Eⱼ as parameters. When values of the elements in the ith row and the jth column of the matrix are calculated, the incident photons correspond to the parameters Eᵢ and Eⱼ, i.e., the parameters Eᵢ and Eⱼ correspond to E and E′ in formula (2). In the formula (2), E denotes the incident photons, E′ denotes a total energy deposition, pᵢ denotes a probability that the incident photons are absorbed in the ith route, and ΔEᵢ denotes an energy deposition generated by the incident photons absorbed in the ith route.

The incident photons may be absorbed in seven routes.

First, the photons may be transmitted through a semiconductor without energy deposition. Correspondingly, the energy deposition ΔEᵢ generated by absorbing the incident photons in this route may be zero. As shown in formula (3), the probability that the incident photons are absorbed in this route may be determined according to formula (4):

$$Δ {E}_{1} = 0 , \qquad ( 3 )$$

$${p}_{1} = exp \{ - {μ}_{t} ( E ) t \} , \qquad ( 4 )$$

wherein μₜ denotes an attenuation coefficient curve of the semiconductor, and t denotes a material thickness condition of the semiconductor.

Second, the photons may be absorbed through a Compton scattering effect. A Compton photon with an energy of E_{c} may be generated and escape a local interaction site. Correspondingly, an energy deposition ΔE₂(θ) generated by the incident photons absorbed at a scattering angle θ of an incident plane in this route may be calculated by formula (5). A probability p₂(θ, ϕ) that the incident photons are absorbed at a scattering angle (θ, ϕ) in this route may be determined according to formula (6):

$$Δ {E}_{2} ( θ ) = E - {E}_{c} ( θ ) , \qquad ( 5 )$$

$${p}_{2} ( θ , ϕ ) = {p}_{c} σ ( θ , ϕ ) {f}_{c} ( {E}_{c} ( θ ) ) , \qquad ( 6 )$$

wherein θ denotes the scattering angle of the incident plane, E denotes the energy of the incident photons, E_{c}(θ) denotes the energy of the escape Compton scattering photon at the scattering angle θ of the incident plane, p_{c} denotes a total probability of Compton scattering, ϕ denotes a scattering amplitude angle of the incident plane, σ(θ, ϕ) denotes a probability of Compton scattering at the scattering angle (θ, ϕ), and ƒ_{c} denotes a function that estimates the probability of the Compton photon escaping a current detector pixel.

Third, the photons may be absorbed through the Compton scattering effect.

The transmitted Compton photons may be locally/partially absorbed. Correspondingly, the energy deposition ΔE₃ generated by absorbing the incident photons in this route may be calculated by a formula (7). A probability p₃(θ, ϕ) that the incident photons are absorbed at the scattering angle (θ, ϕ) in this route may be determined according to formula (8):

$$Δ {E}_{3} = E , \qquad ( 7 )$$

$${p}_{3} ( θ , ϕ ) = {p}_{c} ( 1 - σ ( θ , ϕ ) {f}_{c} ( {E}_{c} ( θ ) ) ) , \qquad ( 8 )$$

wherein θ denotes the scattering angle of the incident plane, E denotes the energy of the incident photons, E_{c}(θ) denotes the energy of the escape Compton scattering photon at the scattering angle θ of the incident plane, p_{c} denotes the total probability of Compton scattering, ϕ denotes the scattering amplitude angle of the incident plane, σ(θ, ϕ) denotes the probability of Compton scattering at the scattering angle (θ, ϕ), and ƒ_{c} denotes the function that estimates the probability of the Compton photon escaping the current detector pixel.

Fourth, the photons may be absorbed through the Compton scattering effect.

The remotely transmitted Compton photons may beabsorbed. Correspondingly, an energy deposition ΔE₄ generated by absorbing the incident photons in this route at the scattering angle θ of the incident plane may be calculated by formula (9). A probability p₄(θ, ϕ) that the incident photons are absorbed at the scattering angle (θ, ϕ) in this route may be determined according to formula (10):

$$Δ {E}_{4} ( θ ) = {E}_{c} ( θ ) , \qquad ( 9 )$$

$${p}_{4} ( θ , ϕ ) = {p}_{c} σ ( θ , ϕ ) {f}_{c} ( {E}_{c} ( θ ) ) , \qquad ( 10 )$$

wherein θ denotes the scattering angle of the incident plane, E_{c}(θ) denotes the energy of the escape Compton scattering photon at the scattering angle θ of the incident plane, p_{c} denotes the total probability of Compton scattering, ϕ denotes the scattering amplitude angle of the incident plane, a(θ, ϕ) denotes the probability of Compton scattering at the scattering angle (θ, ϕ), and ƒ_{c} denotes the function that estimates the probability of the Compton photon escaping the current detector pixel.

Fifth, the photons may be absorbed through a photoelectric effect. K-series fluorescence with an energy Eₖ may be generated and escape a local interaction site. Correspondingly, an energy deposition ΔE₅ generated by absorbing the incident photons in this route may be calculated by formula (11). A probability p₅ that the incident photons are absorbed in this route may be determined according to formula (12):

$$Δ {E}_{5} = E - {E}_{K} , \qquad ( 11 )$$

$${p}_{5} = {p}_{pe} ξ ω {f}_{pe} , \qquad ( 12 )$$

wherein E denotes the energy of the incident photons, E_{K} denotes an energy of the K-series fluorescence, pₚₑ denotes an estimated value of a total probability of the photoelectric effect, a constant ξ denotes a probability of the K-series fluorescence when the energy of the incident photons is E, a constant ω denotes a yield of the K-series fluorescence when the energy of the incident photons is E, and ƒₚₑ denotes an estimated value of a probability that the K-series fluorescence escapes the local interaction site.

Sixth, the photons may be absorbed through the photoelectric effect. No K-series fluorescence may be generated or transmitted K-series fluorescence may be locally absorbed. Correspondingly, an energy deposition ΔE₆ generated by absorbing the incident photons in this route may be determined according to formula (13). A probability p₆ that the incident photons are absorbed in this route may be determined according to formula (14):

$$Δ {E}_{6} = E , \qquad ( 13 )$$

$${p}_{6} = {p}_{pe} ( 1 - ξ ω {f}_{pe} ) , \qquad ( 14 )$$

wherein E denotes the energy of the incident photons, pₚₑ denotes the estimated value of the total probability of the photoelectric effect, the constant ξ denotes the probability of the K-series fluorescence when the energy of the incident photons is E, the constant ω denotes the yield of the K-series fluorescence when the energy of the incident photons is E, and ƒₚₑ denotes the estimated value of the probability that the K-series fluorescence escapes the local interaction site.

Seventh, the photons may be absorbed through the photoelectric effect. Remotely transmitted K-series fluorescence may be absorbed. Correspondingly, an energy deposition ΔE₇ generated by absorbing the incident photons in this route may be calculated by formula (15). A probability p₇ that the incident photons are absorbed in this route may be determined according to formula (16):

$$Δ {E}_{7} = {E}_{K} , \qquad ( 15 )$$

$${p}_{7} = {p}_{c} ξ ω {f}_{pe} , \qquad ( 16 )$$

wherein E_{K} denotes the energy of the K-series fluorescence, p_{c} denotes a total probability of Compton scattering, the constant ξ denotes the probability of the K-series fluorescence when the energy of the incident photons is E, the constant ω denotes the yield of the K-series fluorescence when the energy of the incident photons is E, and ƒₚₑ denotes the estimated value of the probability that the K-series fluorescence escapes the local interaction site.

An estimated value pₚₑ of a total probability of the photoelectric effect may be determined by formula (17):

$${p}_{pe} ( E ) = \frac{{μ}_{pe} ( E )}{{μ}_{t} ( E )} exp \{ - {μ}_{t} ( E ) t \} , \qquad ( 17 )$$

wherein μₚₑ(E) denotes a photoelectric attenuation coefficient of a semiconductor at the energy E, μₜ(E) denotes an attenuation coefficient of the semiconductor at the energy E, μₜ denotes an attenuation coefficient curve of the semiconductor, and t denotes a material thickness combination of the semiconductor.

The function ƒ_{c} that estimates the probability of the Compton photon escaping the current detector pixel may be determined by formula (18):

$${f}_{c} ( {E}_{c} ( θ ) ) \approx exp ( - {μ}_{t} ( {E}_{c} ( θ ) {l}_{eff} ( θ , ϕ ) ) , \qquad ( 18 )$$

wherein θ denotes the scattering angle of the incident plane, ϕ denotes the scattering amplitude angle of the incident plane, E_{c}(θ) denotes the energy of the escape Compton scattering photon at the scattering angle θ of the incident plane, μₜ(E_{c}(θ)) denotes an attenuation coefficient of the semiconductor at the energy E_{c}(θ), μₜ denotes the attenuation coefficient curve of the semiconductor, and I_{eff} denotes an estimated value of an effective route length of the transmitted Compton scattering photon transmitted in a local pixel.

The energy resolution sub-model 420 refers to a model based on the principle of energy spectrum analysis. In some embodiments, the processor may establish the energy resolution sub-model 420 by a statistical simulation method (e.g., Monte Carlo simulation, etc.), which performs statistics of an energy response distribution by considering statistical digital-analog fluctuations of the electron-hole pairs during ionization. The energy resolution sub-model 420 may be characterized as a response function of the PCCT system in the process of energy resolution. For example, the energy resolution sub-model may be constructed by a formula (19):

$${S}_{res} ( {E}^{″} , {E}^{′} ) ∝ exp \{ - \frac{{( {E}^{″} - {E}^{′} )}^{2}}{2 ( F ∈ {E}^{′} + {σ}_{r o}^{2} )} \} , \qquad ( 19 )$$

wherein Sᵣₑₛ denotes the response function (which may be denoted as a matrix) for describing an energy resolution effect, E′ denotes a total energy deposition after processing of an energy deposition sub-model, E″ denotes a resolution energy, F denotes a Fano factor of a semiconductor material, σᵣₒ denotes an energy resolution of an electronic readout device, ∝ denotes a proportional relationship, and exp denotes an exponential function.

The charge sharing sub-model 430 refers to a model based on the effect of charge sharing that occurs between adjacent pixels or channels in the detector. In some embodiments, the processor may establish the charge sharing sub-model 430 by analyzing the charge transmission process of the detector and considering factors such as a structure, an electric field distribution, and diffusion of a charge cloud of the detector. The charge sharing sub-model 430 may be characterized as a response function of the PCCT system in the process of charge sharing. For example, the charge sharing sub-model 430 may be obtained through Monte Carlo simulation, or obtained by fitting through a semi-empirical formula derived based on a physical model.

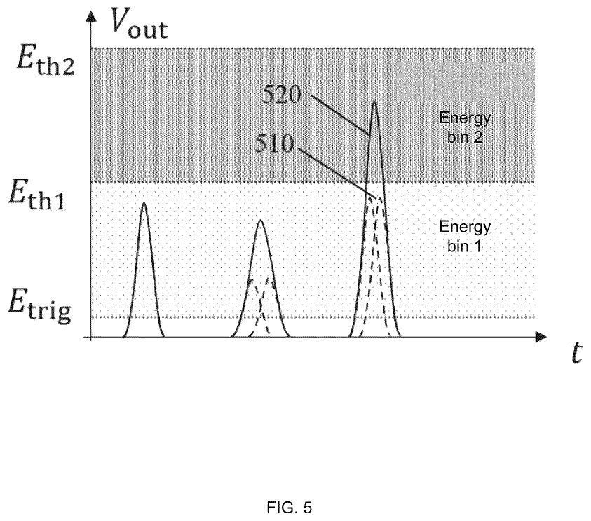

The pulse pileup sub-model 440 refers to a model based on the effect of pulse pileup. In some embodiments, the processor may establish the pulse pileup sub-model 440 based on an arrival time interval of photons, a temporal resolution of the detector, and the shape of a photon pulse, and compensate for one or more effects in the formation of a PCD signal. The pulse pileup sub-model 440 may be characterized as a response function of the PCCT system during the process of pulse pileup. For example, the pulse pileup sub-model 440 may be obtained through Monte Carlo simulation, or obtained by fitting through the semi-empirical formula derived based on the physical model. The form of the semi-empirical formula may be determined based on an electronic readout design and parameters of the PCD. More descriptions for the pulse pileup sub-model 440 may be found elsewhere in the present disclosure (e.g., FIGS. 5-8 and the descriptions thereof).

The energy binning sub-model 450 refers to a model based on the absorption features of matter for photons of different energies. In some embodiments, the processor may obtain a response relationship between the detector and photons of different energies through experimental data. The energy binning sub-model 450 may be established by setting an energy binning threshold according to requirements or experimental conditions and defining an energy binning strategy. The energy binning sub-model 450 may be characterized as a response function of the PCCT system model in the process of energy binning. For example, the energy binning sub-model may be energy binning constructed by a formula (20):

$${S}_{bin} ( k , {E}_{peak} ) = \{ \begin{matrix}1 , & {T}_{k} \leq {E}_{peak} \leq {T}_{k + 1} \\ 0 , & otherwise\end{matrix} , \qquad ( 20 )$$

wherein S_{bin} denotes the response function (which may be denoted as a matrix) describing the process of energy binning, k denotes a kth energy bin, Eₚₑₐₖ denotes a scaled waveform peak energy, Tₖ denotes a lower threshold of the kth energy bin, Tₖ₊₁ denotes an upper threshold of the kth energy bin, and otherwise denotes other situations.

In some embodiments, a PCCT system model may be formed by the plurality of sub-models through cascading. For example, the PCCT system model may be represented by formula (21).

$$y = {S}_{bin} {S}_{PP} {S}_{CS} {S}_{res} {S}_{abs} {T}_{phan} {T}_{filter} {S}_{source} , \qquad ( 21 )$$

wherein S_{bin} denotes a response function of the PCCT system model in a process of energy binning, S_{PP} denotes a response function of the PCCT system model in a process of pulse pileup, S_{CS} denotes a response function of the PCCT system model in a process of charge sharing, Sᵣₑₛ denotes a response function of the PCCT system model in a process of energy resolution, and S_{abs} denotes a response function of the PCCT system model in a process of energy resolution energy deposition. S_{source} denotes an incident spectrum from the X-ray source, and determined by the scanning parameters of the X-ray source. Tₚₕₐₙ denotes an energy-dependent transmissivity projected through the plurality of phantoms. T_{filter} denotes an energy-dependent transmissivity projected through a collimator (also referred to as a filter) and is configured to modulate X-ray attenuation. In some embodiments, the filter may include a filter design before and/or after the target subject, such as a filter (e.g., a butterfly filter, a planar filter, a wedge filter, a bowtie filter, etc.) on the X-ray source side and/or anti-scatter grid located between the detector and the subject to be scanned.

In some embodiments, Tₚₕₐₙ and T_{filter} are represented by formula (22) and formula (23), respectively.

$${T}_{phan} = D [ exp ( - {μ}_{phan} A {ρ}_{phan} ) ] , \qquad ( 22 )$$

$${T}_{filter} = D [ exp ( - {μ}_{filter} A {ρ}_{filter} ) ] , \qquad ( 23 )$$

wherein μₚₕₐₙ denotes a material mass attenuation function of the phantom, ρₚₕₐₙ denotes a composition map of the phantom, μ_{filter} denotes a material mass attenuation function of the filter, ρ_{filter} denotes a composition map of the filter, and A denotes a forward projection operator.

In some embodiments, an incident energy spectrum Sᵢₙ of the PCD system may be represented by formula (24):

$${S}_{in} = {T}_{phan} {T}_{filter} {S}_{source} , \qquad ( 24 )$$

wherein Tₚₕₐₙ denotes a projection transmissivity changing with the energy through the phantom, T_{filter} denotes a projection transmissivity changing with the energy through the filter, and S_{source} denotes the source spectrum from the X-ray source. The incident energy spectrum Sᵢₙ refers to an energy spectrum formed by X-rays after passing through one or more incident objects that irradiate the detector.

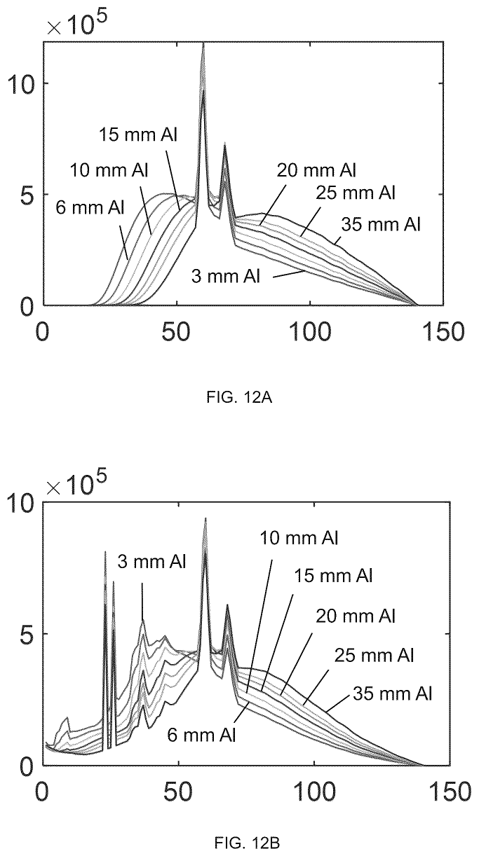

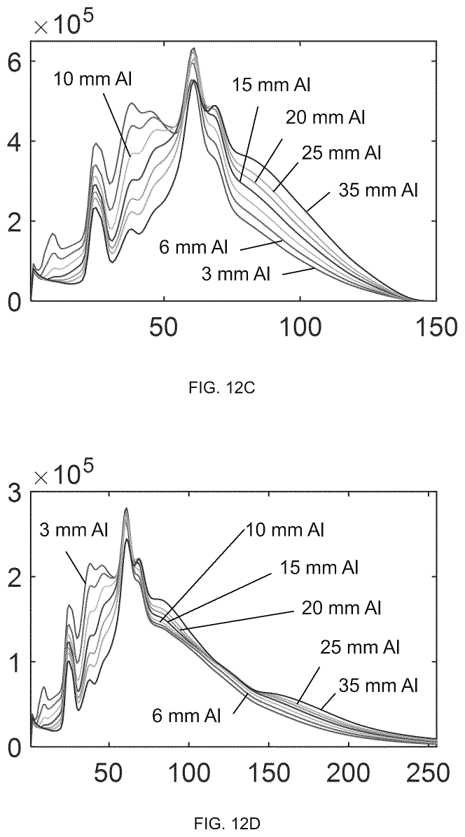

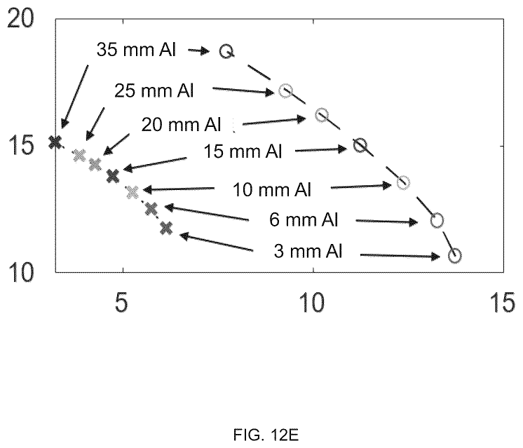

The response of the detector at each stage may be parameterized by corresponding sub-models through the PCCT system model formed by cascading of the energy deposition sub-model, the energy resolution sub-model, the charge-sharing sub-model, the pulse pileup sub-model, and the energy binning sub-model, and the resulting PCCT system model may be the physical model based on the photon transmission path, which may gradually eliminate or partially eliminate the effects of energy deposition, energy resolution, charge sharing, pulse pileup, and energy binning on photon counting, thus improving the accuracy of the PCCT system model. For example, as shown in FIG. 12A-FIG. 12E, FIG. 12A is a schematic diagram illustrating an incident spectrum (i.e., a complete energy spectrum (Sᵢₙ) of incident photons after radiation rays emitted by a radiation source pass through the phantoms before entering a detector) obtained by testing seven phantoms of 3 mm AI-35 mm Al. A horizontal axis represents the incident energy of the incident photons in kilo-electron-volts (keV), and a vertical axis represents the intensity of photons corresponding to different energy points of the incident photons, measured in counts per second per kilo-electron volt (cps/keV). FIG. 12B is a schematic diagram illustrating an energy spectrum S_{abs} after processing according to the energy deposition sub-model according to some embodiments of the present disclosure, and describes the energy deposition situation of photons in a material. A horizontal axis represents a deposited energy in keV, and a vertical axis represents an intensity of photons corresponding to different deposited energy points, measured in cps/keV. FIG. 12C is a schematic diagram illustrating an energy spectrum (SᵣₑₛS_{abs}Sᵢₙ) after processing based on an energy deposition sub-model and an energy resolution sub-model according to some embodiments of the present disclosure. A horizontal axis represents resolved energy values in keV, and a vertical axis represents an intensity of photons corresponding to different resolved energy values, measured in cps/keV. It may be observed that the energy resolution in the energy spectrum is decreased. FIG. 12D is a schematic diagram illustrating an energy spectrum (S_{PP}S_{CS}SᵣₑₛS_{abs}Sᵢₙ) after processing based on an energy deposition sub-model, an energy resolution sub-model, a charge sharing sub-model, and a pulse pileup sub-model according to some embodiments of the present disclosure. A horizontal axis represents a pixel readout energy in keV, and a vertical axis represents an intensity of photons corresponding to different pixel readout energy values, measured in cps/keV. It may be observed that the blurring effect of charge diffusion and the counting rate are relatively reduced. FIG. 12E is a schematic diagram illustrating an energy spectrum (S_{bin} S_{PP}S_{CS}SᵣₑₛS_{abs}Sᵢₙ) after processing based on an energy deposition sub-model, an energy resolution sub-model, a charge sharing sub-model, a pulse pileup sub-model, and an energy binning sub-model according to some embodiments of the present disclosure. A processor may determine the photon counting data based on different energy ranges. A horizontal axis represents photon counting data corresponding to an energy range of an energy bin Bin 1, measured in mega-counts per second (Mcps), and a vertical axis represents photon counting data corresponding to an energy range of an energy bin Bin 2, measured in Mcps. A circle symbol indicates the photon counting data without pulse pileup, while a cross symbol indicates the photon counting data with pulse pileup. FIG. 12E also illustrates the influence of the material thickness of a phantom on the photon counting data.

It should be noted that the foregoing description of the PCCT system model including the plurality of sub-models in cascade is intended to be exemplary and illustrative only, and does not limit the scope of application of the present disclosure. Those skilled in the art may make various corrections and changes to the PCCT system model and sub-models thereof under the guidance of the present disclosure.

However, these corrections and changes remain within the scope of the present disclosure.

FIG. 5 is a schematic diagram illustrating an exemplary pulse pileup effect according to some embodiments of the present disclosure. As shown in FIG. 5, a transverse axis t represents a time axis, a vertical axis Vₒᵤₜ represents energy, E_{trig} represents threshold energy triggering photon counting, Eₜₕ₁ represents an upper threshold of a low-energy bin (e.g., an energy bin 1), and a lower threshold of a high-energy bin (e.g., an energy bin 2), and Eₜₕ₂ represents an upper threshold of the high-energy bin (e.g., the energy bin 2). E_{trig} also represents a lower threshold of the low-energy bin (e.g., the energy bin 1).

As shown in FIG. 5, a waveform 510 of photon counting may include a real pulse curve 510 and an observed pulse curve 520. The real pulse curve 510 may include two pulses superimposed next to each other on the time axis, which may be incorrectly counted as one pulse in the observed pulse curve 520. In addition, the pulse amplitude of the incorrectly counted pulse may be close to the sum of the pulse amplitudes of the two pulses in the real pulse curve, which may cause a plurality of counts that should be counted in the low-energy bin (e.g., the energy bin 1) are incorrect counted as a single count in the high-energy bin (e.g., the energy bin 2).

Therefore, in some embodiments of the present disclosure, a pulse pileup effect in a non-paralytic PCD may be simulated through formulas (25)-(29):

$$y ( E ; α ) = \frac{α}{1 + α τ} {\sum }_{p} {y}_{p} ( E ; α ) , \qquad ( 25 )$$

$${y}_{p} ( E ; α ) = P {r}_{p} ( α ) {S}_{p} ( E ) , \qquad ( 26 )$$

$${Pr}_{p} ( α ) = \frac{exp ( - α τ ) {( α τ )}^{p}}{p !} , \qquad ( 27 )$$

$${S}_{0} ( E ) = \frac{S ( E )}{\int S ( E ) d E} , \qquad ( 28 )$$

$${S}_{p} ( E ) = {S}_{0} ( E ) * {S}_{p - 1} ( E ) , \qquad ( 29 )$$

wherein E denotes photon energy, a denotes the incident count rate of the PCD in Mcps, which represents a photon statistical property of the PCD in the formulas 25-29; y(E; α) denotes a total count rate at the photon energy E after pulse pileup when the incident count rate is α; p denotes a pulse pileup order; μₚ(E, α) denotes a normalized contribution of pth pulse pileup at the incident count rate of α, which is obtained by multiplying a probability Prₚ(α) of the pth pulse pileup at the incident count rate of α and a normalized output energy spectrum S_{P}(E) generated by the pth pulse pileup; S(E) denotes an incident energy spectrum of the PCD; S₀(E) denotes a normalized incident energy spectrum; τ denotes an effective deadtime in μsec, and exp denotes an exponential function.

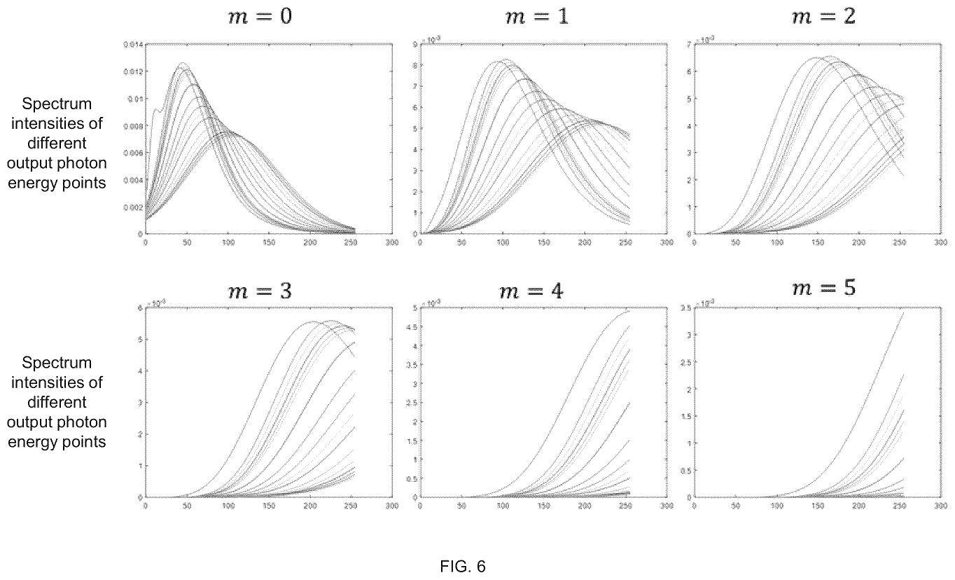

FIG. 6 is a schematic diagram illustrating output energy spectra (i.e., a normalized contribution yₚ(E) of a pth pulse pileup) generated by each order of pulse pileup determined based on a formula (29) when a PCD receives different incident energy spectra (140 kVp typical X-ray source energy spectra pass through different thicknesses of aluminum for filtration). As shown in FIG. 6, a vertical axis represents energy spectrum intensities of different output photon energy points, and a transverse axis represents an energy photon energy in keV. As shown in FIG. 6, an equivalent output energy spectrum generated by each order of pulse pileup effect may depend on the intensity and shape of an incident energy spectrum.

In some embodiments, a PCCT system model may include at least one energy spectrum formation model (i.e., spectrum model for brevity) corresponding to each of at least one energy bin. In some embodiments, the spectrum model corresponding to each of at least one energy bin may include a plurality of model parameters under a plurality of different pulse pileup orders. The plurality of model parameters under the plurality of different pulse pileup orders corresponding to one energy bin may form a group of model parameters. The spectrum model corresponding to an energy bin may characterize a mapping relationship between a phantom parameter (e.g., the material line integral, the thickness), one or more scanning parameters, and photon counting data corresponding to the energy bin via the group of model parameters.

In some embodiments, the calibrated PCCT system model may include target values of the group of model parameters corresponding to each of the at least one energy bin under different pulse pileup orders.

The energy bin refers to an energy interval. Different energy bins may correspond to different energy interval thresholds. Photons may be categorized into corresponding energy bins based on the energy of photons, and interval photon counting data may be separately recorded for each energy bin. A collection of the interval photon count data of each energy bin may present an energy distribution of the incident photons. The energy interval thresholds may be the default setting of the system 100 or set by an operator of the system.

In some embodiments, each energy bin may correspond to a corresponding spectrum model (i.e., an energy spectrum counting forming function, spectrum formation function for brevity) that describes an energy distribution of photons within the energy bin.

The pulse pileup order describes the extent of the pulse pileup effect. For example, if there is no pulse pileup effect when counting incident photons, the pulse pileup order may 0. If pulses of two incident photons are counted as a single photon, the pulse pileup order may be 1. The higher the pulse pileup order, the more severe the pulse pileup effect, and the greater the error in the photon counting data.

For each energy bin, each of the plurality of pulse pileup orders may correspond to corresponding model parameters.

The spectrum model corresponding to an energy bin may be configured to describe the energy response of the incident photons at different pulse pileup orders. The spectrum model corresponding to the energy bin may include one or more model parameters under each of the different pulse pileup orders. In some embodiments, the spectrum model corresponding to the energy bin may include multiple spectrum sub-models (also referred to as energy spectrum counting forming sub-functions, i.e., spectrum formation sub-function for brevity) each of which corresponds to one of the different pulse pileup orders. A spectrum formation sub-function corresponding to one of the different pulse pileup orders may be denoted by the one or more model parameters under the pulse pileup order. The spectrum formation sub-function corresponding to the pulse pileup order may be configured to provide a mapping relationship between photon counting data under the pulse pileup order, the one or more scanning parameters, and a reference material parameter through the model parameters under the pulse pileup order. For example, FIG. 7A is a schematic diagram illustrating an exemplary spectrum model according to some embodiments of the present disclosure. As shown in FIG. 7A, the spectrum model corresponding to the energy bin may include multiple spectrum formation sub-functions 710, 720, . . . , N corresponding to different pulse pileup orders 0, 1, . . . , p. When the pulse pileup order is 1, the energy of the incident photons may be determined by the spectrum formation sub-function 720, which corresponds to the pulse pileup order 1 when the photons are incident onto the detector. Moreover, the photon counting data for the energy bin b₁ under the pulse pileup order 1 may be determined according to the spectrum formation sub-function 710.

In some embodiments, for each energy bin, the spectrum model may be obtained by adding the spectrum formation sub-models under the plurality of different pulse pileup orders.

In some embodiments, each energy bin may correspond to the group of model parameters. In some embodiments, the group of model parameters may include a plurality of spectrum generation parameters under different pulse pileup orders. For example, for the energy bin b₁, the set of spectrum generation parameters may include S_{1,b1} . . . S_{N,b1} under pulse pileup orders 1-N. N may be an integer of 2, 3, 8, etc.

In some embodiments, a spectrum formation sub-model under a pulse pileup order may include a product of the one or more model parameters under the pulse pileup order, a tube current, and an exponential function whose base is a natural constant. An exponent of the exponential function may be an opposite number of the material line integral.

More descriptions regarding the model parameters, the material line integral, and the scanning parameter may be found in FIG. 2 and related descriptions thereof.

In some embodiments, the spectrum model corresponding to an energy bin may be represented by a formula (30):

$${y}_{b} = f ( α , τ ) I {\sum }_{p = 0 , 1 , 2 … N} {( α τ )}^{p} {S}_{p , b} exp ( - μ L ) , \qquad ( 30 )$$

where y_{b} is the photon counting data of a bth energy bin generated by a detector pixel; α is an incident count rate; τ is detector effective dead time; ƒ(α, τ) is a counting loss correction function related to α and τ; I is a tube current of scanning parameters; b is an identifier or serial number of an energy bin; p is the pulse pileup order; S_{p,b} is a spectrum generation parameter corresponding to the bth energy bin under the pulse pileup order p, and N is the maximum value of the pulse pileup order; L is the material thickness of a reference material (e.g., the material of a phantom), μ is attenuation coefficient of the reference material (e.g., the material of a phantom), and a product of the material thickness L and the attenuation coefficient curve μ is the material line integral; and exp represents the exponential function. The effective dead time τ may be set as a nominal dead time or may be calibrated through model fitting. The effective dead time τ and/or an incident count rate may be spectrum influence parameters. In some embodiments, if the reference material is determined, μ may be determined. If another reference material is used to represent a subject, a factor may be multiplied by the attenuation coefficient of the reference material to modify the spectrum model.

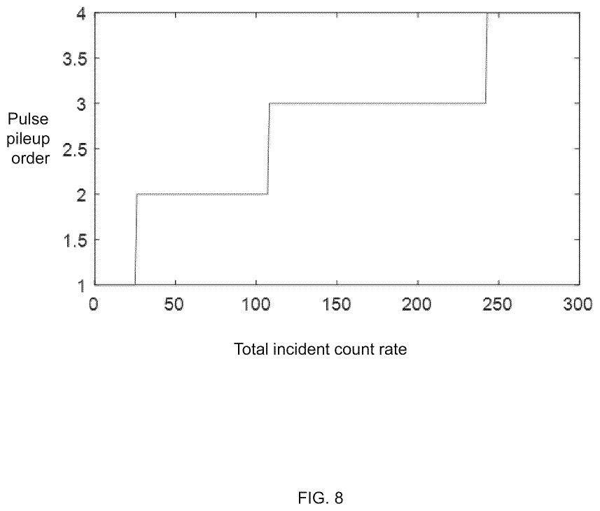

In sone embodiments, the maximum value of the pulse pileup order may be determined based on a total incident count rate. FIG. 8 is a schematic diagram illustrating a changing trend in the maximum value of the pulse pileup order that contributes more than 1‰ to a total contribution to an output signal under different total incident count rates according to some embodiments of the present disclosure. A vertical axis represents a pulse pileup order, and a transverse axis represents a total incident count rate. The total incident count rate may be used to determine the proportion of each order of pile-up event. The total incident count rate may be forwardly simulated.

As shown in FIG. 8, when the total incident count rate is larger, such as from 0 to 300 Mcps, the effective pulse pileup order may increase from 0 to 4. In some embodiments, the highest order (i.e., a maximum value N of the pulse pileup order) of a semi-empirical formula under different total incident count rates may be determined according to the changing trend (e.g., the curve in FIG. 8) in the highest pulse pileup order that contributes more than 1‰ to the total contribution to the output signal under different total incident count rates.

In some embodiments, the counting loss correction function ƒ(α, τ) may be estimated from a total count of the same material thicknesses L under different tube currents I. In some embodiments, the counting loss correction function ƒ(α, τ) may be a polynomial related to the dead time and the incident count rate. For example, the counting loss correction function ƒ(α₀, τ) may be represented by the following formula (31):

$$f ( {α}_{0} , τ ) = \frac{exp ( - {α}_{0} τ )}{1 + {α}_{0} τ} , \qquad ( 31 )$$

where α₀ is the incident count rate without pulse pileup, τ is the dead time of the detector, and exp represents the exponential function.

In some embodiments, the function (31) may be determined based on the ratio between mA normalized measurements acquired at low-mA and high-mA. For example, an equation can be constructed to obtain a counting result by substituting the material thickness L and the tube current I into the equation several times, respectively. The material thickness is the same each time the equation is substituted, but the tube current is different. In some embodiments, the function (31) may have a form of exp(−ατ), 1/(1+ατ), or a polynomial of α and τ.

In some embodiments of the present disclosure, as a result of scanning the plurality of phantoms, combining the experimental results and the plurality of phantoms and the scanning parameters used in the scanning, the incident count rate, the tube current, the attenuation coefficient, and the material thicknesses may be important factors affecting photon counting, which may be taken as at least one parameter of the spectrum model in the formula (30). Using the spectrum model, the plurality of model parameters affecting photon counting during the photon transmission path may be compressed, which is conducive to obtaining sparser spectrum parameters. Through the spectrum model in the formula (30), not only is it conducive to simplifying the PCCT system model, but it also takes into account different influences that the pulse pileup effects of different orders may have on photon counting, and forms the spectrum model of the energy response spectrum that include the plurality of different pulse pileup orders, and the resulting PCCT system model may eliminate the counting error caused by pulse pileup as much as possible.

In some embodiments, the spectrum parameters may be determined by a fitting algorithm based on the spectrum model, a plurality of sets of photon counting data of a plurality of phantoms, the scanning parameters, and the phantom parameters of the plurality of phantoms.

In some embodiments, the count of the plurality of sets of photon counting data may be greater than the maximum value of the pulse pileup order.

In some embodiments of the present disclosure, the model parameters may be obtained more easily and quickly by calculating and determining the spectrum parameters through substituting the photon counting data, the scanning parameters, and the material line integrals into the spectrum model.

FIG. 7B is a flowchart illustrating a process of calibrating a spectrum model corresponding to any energy bin according to some embodiments of the present disclosure. In some embodiments, process 700 may be executed by the electronic component 110-3. For example, the process 700 may be stored in a storage device (e.g., the storage device 110-4) in the form of a program or instruction, and when the electronic component 110-3 executes the program or instructions, the process 700 may be implemented. The schematic diagram illustrating the operation of the process 700 below is illustrative. In some embodiments, the process may be completed using one or more additional operations not described above and/or one or more operations not discussed above. As shown in FIG. 7B, the process 700 may include the following operations. In addition, the order of operations of the process 700 is shown in FIG. 7B and described below are non-limiting.

In 702, for a specific energy bin of at least one energy bin, determining initial values of model parameters corresponding to different pulse pileup orders.

In some embodiments, the model parameters may include spectrum generation parameters and spectrum influence parameters. The spectrum generation parameters may be related to at least one process of X-ray generation, X-ray absorption, energy deposition, energy resolution, charge sharing, a pulse pileup effect, and energy binning in a generation process of photon counting data. The spectrum influence parameters may be related to the response of the detector. For example, the spectrum influence parameters may include a performance parameter of the detector, such as the dead time, the incident count rate, the detection efficiency, etc.

The initial values of the model parameters refer to model parameters to be optimized. For example, the initial values of the model parameters may include initial values of the spectrum generation parameters and/or initial values of spectrum influence parameters.

As a further example, the initial values of the spectrum generation parameters may include an initial value of the spectrum generation parameter when the pulse pileup order is 0 denoted as S′_{0,b}, an initial value of the spectrum generation parameter when the pulse pileup order is 1 denoted as S′_{1,b}, . . . , an initial value of the spectrum generation parameter when the pulse pileup order is p denoted as S′_{p,b}.

As a still further example, the initial values of the spectrum influence parameters may include an initial value of an incident count rate when the pulse pileup order is 0 denoted as α′₀; an initial value of the incident count rate when the pulse pileup order is 1, denoted as α′₁, . . . , an initial value of the incident count rate when the pulse pileup order is p denoted as α′ₚ.

In some embodiments, the initial value of the spectrum influence parameter (e.g., the incident count rate, the dead time) corresponding to different pulse pileup orders may be the same. In some embodiments, the target value of the spectrum influence parameter (e.g., the incident count rate, the dead time) corresponding to different pulse pileup orders under the specific energy bin may be the same.

In some implementations, the processor may determine the initial values of the model parameters based on a Monte Carlo simulation algorithm.

In some embodiments, the processor may determine the initial values of the model parameters based on a plurality of sets of photon counting data obtained by scanning a plurality of phantoms. Each set of the plurality of sets of photon counting data may be acquired by a photon counting computed tomography (PCCT) device scanning one of a plurality of phantoms according to one or more scanning parameters. More descriptions for the plurality of sets of photon counting data maybe found elsewhere in the present disclosure (e.g., FIG. 2 and the descriptions thereof).

In some embodiments, the processor may determine a reference set of photon counting data (also referred to as a reference photon counting set) among the plurality of sets of photon counting data; and divide the reference photon counting set into reference photon counting subsets corresponding to different energy bins. For the specific energy bin of different energy bins, the processor may determine the initial values of the model parameters under the specific energy bin based on the reference photon counting subset under the specific energy bin.

In some embodiments, the reference photon counting set may refer to a set of photon counting data acquired by scanning a phantom according to a reference tube current that satisfies a condition. In some embodiments, the reference tube current satisfying the condition may include that the reference tube current is minimum among tube currents for acquiring the plurality of sets of photon counting data. In some embodiments, the reference tube current satisfying the condition may include that the reference tube current is less than a current threshold (e.g., 20 mA, 30 mA, etc.).

In some embodiments, for the specific energy bin, the processor may determine, based on the reference photon counting subset under the specific energy bin, a reference model parameter; and determine, based on the reference model parameter, the initial value of a model parameter under any pulse pileup order.

The reference model parameter may include a spectrum generation parameter when the pulse pileup order is zero and the reference tube current satisfies the condition. The reference model parameter may be applied to the reference tube current, but not applied to other tube current. In other words, a spectrum model including the reference model parameter (also referred to as a reference spectrum model) may provide a mapping relationship between photon counting data under 0 pulse pileup order acquired based on the reference tube current and the material parameter of a reference material.

In some embodiments, the processor may determine, based on the reference spectrum model corresponding to the reference tube current, the reference photon counting subset of a phantom under the specific energy bin, and the material thickness and the attenuation coefficient of the phantom, the reference spectrum generation parameter S_{ref,b} (i.e., the reference model parameter). The reference spectrum model may be denoted by a formula (32):

$${y}_{ref} = {S}_{ref , b} exp ( - {μ}_{1} {L}_{1} ) , \qquad ( 32 )$$

wherein y_{ref} denotes a reference photon count subset obtained by scanning the phantom A without the influence of the pulse pileup effect (i.e., the pulse pileup order is zero), μ₁ denotes the attenuation coefficient of the phantom A, L₁ denotes the thickness of the phantom A, a product of the material thickness L₁ and the attenuation coefficient curve p₁ is the material line integral of the phantom, and S_{ref,b} denotes the reference model parameter. S_{ref,b} may be determined according to formula (32) based on y_{ref}, L₁ and μ₁.

In some embodiments, the processor may determine multiple reference photon counting sets from the plurality of sets of photon counting data, and the tube currents for acquiring the multiple reference photon counting may satisfy the condition. The processor may determine multiple reference model parameters each of which is determined based on one of the multiple reference photon counting sets according to operations as described above, e.g., according to formular (32). A final reference model parameter may be determined based on the multiple reference model parameters. For example, an average of the multiple reference model parameters may be designated as the final reference model parameter. As another example, a median of the multiple reference model parameters may be designated as the final reference model parameter.

In some embodiments, the processor may determine, based on the reference model parameter, an initial value of the model parameter corresponding to any pulse pileup order. In some embodiments, the processor may determine, based on the reference model parameter and a preset comparison table, the initial value of the model parameter corresponding to any pulse pileup order. The preset comparison table may include a correspondence relationship between the reference model parameters and the initial value of the model parameter corresponding to any pulse pileup order. The preset comparison table may be preset based on historical data and prior knowledge. The processor may query, based on the currently determined reference model parameter, similar or close reference model parameters in the preset comparison table, and determine the initial value of the model parameter corresponding to any pulse pileup order as the initial value of the model parameter corresponding to any pulse pileup order corresponding to the currently determined reference model parameter.

In some embodiments, for the specific energy bin, when a pulse pileup order is zero, the processor may determine the initial value of the model parameter corresponding to the pulse pileup order of 0 by normalizing the reference model parameter based on the reference tube current. For example, the initial value of the model parameter corresponding to the pulse pileup order of 0 may be determined according to formula (33) and formula (34):

$${y}_{b} = {lim}_{α τ \to 0} \frac{exp \{ - α τ \}}{1 + α τ} I {\sum }_{p} {( α τ )}^{p} {S}_{p , b} exp \{ - μ L \} = I {S}_{0 , b}^{′} exp \{ - μ L \} = \frac{I}{{I}_{ref}} {S}_{ref , b} exp \{ - μ L \} , \qquad ( 33 )$$

$${S}_{0 , b}^{′} = \frac{1}{{I}_{ref}} {S}_{ref , b} , \qquad ( 34 )$$

wherein S′_{0,b} denotes the initial value of the spectrum generation parameter corresponding to the bth energy bin when the pulse pileup order is zero; S_{ref,b} denotes the reference model parameter, I denotes the tube current and I_{ref} denotes the reference tube current.

In some embodiments, for the specific energy bin, when a pulse pileup order is non-zero, the processor may determine the initial value of the spectrum generation parameter corresponding to the pulse pile order being zero by normalizing the reference model parameter based on the reference tube current; and determine, based on the initial value of the spectrum generation parameter corresponding to the pulse pile order being of zero, the initial value of the spectrum generation parameter corresponding to the pulse pile order being of non-zero.

More descriptions regarding determining the initial value of the spectrum generation parameter corresponding to the pulse pileup order being zero may be found in the previous descriptions.

In some embodiments, the processor may determine, based on a relationship between the initial value of the spectrum generation parameter corresponding to the pulse pileup order being of zero and the initial value of the spectrum generation parameter corresponding to the pulse pileup order being of non-zero, the initial value of the spectrum generation parameter corresponding to the pulse pileup order being of non-zero on the basis that the initial value of the spectrum generation parameter corresponding to the pulse pileup order being of zero is determined. For example, the relationship between the initial value of the spectrum generation parameter corresponding to the pulse pileup order being zero and the initial value of the spectrum generation parameter corresponding to the pulse pileup order being of non-zero may be a certain proportional relationship. After the initial value of the spectrum generation parameter corresponding to the pulse pileup order being of zero is determined, the initial value of the spectrum generation parameter corresponding to the pulse pileup order being of non-zero may be determined based on the proportional relationship. Merely by way of example, the initial value of the spectrum generation parameter corresponding to the pulse pileup order of non-zero may be 1/a (e.g., 1/10, ⅕, ½, etc.) of the initial value of the spectrum generation parameter when the pulse pileup order being of 0. If the initial value of the spectrum generation parameter corresponding to the pulse pileup order being of zero is determined as “s”, it is determined that the initial value of the spectrum generation parameter corresponding to the pulse pileup order being of non-zero may be “s/a”.

In some embodiments, the initial values of spectrum generation parameters corresponding to different pulse pileup orders being of non-zero may be different. The ratio of the initial values of spectrum generation parameters corresponding to different pulse pileup orders being of non-zero to the initial value of the spectrum generation parameter corresponding to the pulse pileup orders being of zero may be a default setting of the system or set by the operator of the system 100.

In some embodiments, the initial values of spectrum generation parameters corresponding to different pulse pileup orders being non-zero may be the same.

In some embodiments, the relationship between the initial value of the spectrum generation parameter corresponding to the pulse pileup order being zero and the initial value of the spectrum generation parameter corresponding to the pulse pileup order being non-zero may be preset based on historical data or prior knowledge.

In some embodiments, the processor may determine, based on the reference model parameter, and the material thickness and the attenuation coefficient of the phantom, an initial value of the spectrum influence parameter corresponding to one of different pulse pileup orders. For example, the processor may determine the initial value of the spectrum influence parameter according to formula (35):

$${α}_{0}^{′} = \frac{I}{{I}_{ref}} \times {S}_{ref , all bin} \times exp ( - µ L ) , \qquad ( 35 )$$

wherein α₀′ denotes the initial value of the spectrum influence parameter corresponding to any energy bin; S_{ref,all bin} denotes a sum of reference model parameters corresponding to a plurality of energy bins, L denotes the material thickness of a phantom, I_{ref} denotes the reference tube current, and μ denotes the attenuation coefficient of the material of the phantom. The phantom may be used to acquire the reference photon counting subsets for determining the reference model parameters corresponding to a plurality of energy bins. In some embodiments, the initial values of the spectrum influence parameters corresponding to different energy bins may be the same. In some embodiments, the initial values of the spectrum influence parameters corresponding to different pulse pileup orders under the same energy bin may be the same. In some embodiments, the initial values of the spectrum influence parameters corresponding to different pulse pileup orders under the same energy bin may be different. The initial values of the spectrum influence parameters corresponding to pulse pileup orders being of non-zero may be determined based on the initial value of the spectrum influence parameter corresponding to the pulse pileup order being of zero.

In some embodiments of the present disclosure, the calibration accuracy can be effectively improved by determining the initial value of the model parameter through the initial non-pileup spectrum model of the low tube current; and the initial value of the model parameter when the pulse pileup order is non-zero can be rapidly determined through the initial value of the model parameter when the pulse pileup order is zero and the pulse pileup order.

In 704, determining target values of the model parameters corresponding to different pulse pileup orders, based on the initial values of the model parameters corresponding to the different pulse pileup orders.

In some embodiments, for the specific energy bin, the processor may determine, based on the initial values of the model parameters of the spectrum model corresponding to the energy bin under the different pulse pileup orders, the target values of the model parameters.

The target value of the model parameter refers to a model parameter obtained after optimization. In some embodiments, the target value of the model parameter under a pulse pileup order may include a target value of the spectrum generation parameter and/or a target value of a spectrum influence parameter under the pulse pileup order.

In some embodiments, the target value of the spectrum generation parameter may include the target value of the spectrum generation parameter when the pulse pileup order is 0 denoted as S_{0,b}, the target value of the spectrum generation parameter when the pulse pileup order is 1 denoted as S_{1,b}, . . . , the target value of the spectrum generation parameter when the pulse pileup order is p denoted as S_{p,b}.

In some embodiments, the target value of the spectrum influence parameter may include a target value of an incident count rate when the pulse pileup order is 0 denoted as α₀; the target value of the spectrum parameter when the pulse pileup order is 1 denoted as α₁, . . . , the target value of the spectrum parameter when the pulse pileup order is p denoted as α₁ₚ.

In some embodiments, the processor may determine the target value of the model parameter by optimizing the initial value of the model parameter through an optimization algorithm. The optimization algorithm may include an iterative algorithm for solving the optimization problem, such as the maximum likelihood expectation method (MLEM), gradient descent, a genetic algorithm, or the like.

In some embodiments, an iterative process including multiple iterations may be performed to determine the target values of the model parameters. In some embodiments, for the specific energy bin, in each iteration, the processor may determine, based on the spectrum model including the initial values of the model parameters, and a phantom parameter and one or more scanning parameters corresponding to one set of the plurality of sets of photon counting data, a photon counting estimation corresponding to a photon counting subset, among the set of photon counting data, corresponding to the specific energy bin; construct, based on an error between the photon counting subset and the photon counting estimation, a target optimization function; in response to determining that a termination condition is not satisfied, update the initial values of the model parameters based on the target optimization function to determine updated values of the model parameters and designate the updated values of the model parameters as the initial values of the model parameters used in the next iteration; and in response to determining that the termination condition is satisfied, determine the initial values of the model parameters generated in the current iteration as the target values of the model parameters. The initial values of the model parameters in the first iteration of the iterative process may be the initial values determined in operation 702.

The photon counting estimation refers to photon counting data estimated based on the spectrum model corresponding to the energy bin. The photon counting measurement refers to photon counting data obtained by scanning the phantom using the PCCT device.

In some embodiments, the processor may divide the set of photon counting data into a plurality of subsets of photon counting data (i.e., photon counting subsets) corresponding to different energy bins to obtain the photon counting subset corresponding to the specific energy bin.

In some embodiments, for one set of the plurality of sets of photon counting data, the processor may obtain the photon counting estimation under the specific energy bin by substituting the initial values of the model parameters, the phantom parameter (e.g., the material thickness, and the attenuation coefficient curve of the phantom) and the scanning parameter (e.g., the tube current) corresponding to the set of photon counting data, and the photon counting subset under the specific energy bin into the spectrum model corresponding to the specific energy bin.

Merely by way of example, for a photon counting subset y_{b}, cali (I, L, μ, S_{p,b}′) corresponding to a specific energy bin, the processor may obtain the photon counting estimation y̅_̅{̅b̅}̅(I, L, μ, S_{p,b}′) by substituting the initial value S_{p,b} of the model parameter, the material thickness L, the attenuation coefficient curve μ, and the tube current I corresponding to the photon counting subset into the spectrum model (e.g., the formula (30)).

In some embodiments, in each iteration, for each set of the plurality of sets of photon counting data, the processor may determine the error between the photon counting subset and the photon counting estimation corresponding to the photon counting subset, determine a sum of errors each of which is the error between the photon counting subset and the photon counting estimation corresponding to one set of the plurality of sets of photon counting data, and construct a target optimization function based on the sum of the errors. In other words, the target optimization function may constrain the total error (i.e., the sum of errors each of which is between the photon counting subset and the photon counting estimation corresponding to one set of the plurality of sets of photon counting data).

In some embodiments, in each iteration, for one set of the plurality of sets of photon counting data, the processor may determine the error between the photon counting subset and the photon counting estimation corresponding to the photon counting subset, and construct a target optimization function based on the error between the photon counting subset and the photon counting estimation corresponding to the set of the photon counting data. In other words, the target optimization function may constrain the error between the photon counting subset and the photon counting estimation corresponding to the set of the photon counting data. For example, the target optimization function may be constructed by a formula (36):

$${\hat{S}}_{p , b} = \backslash arg \backslash {min}_{\{ {S}_{p , b} \}} {\sum }_{L} {\sum }_{I} \vert {y}_{b} , cali ( I , L , µ , {S}_{p , b}^{′} ) - \overline{{y}_{b}} ( I , L , µ , {S}_{p , b}^{′} ) {\vert}_{2}^{2} , \qquad ( 36 )$$

where S_{p,b}′ denotes the initial value (e.g., the initial value of the spectrum generation parameter of an energy bin corresponding to each pulse pileup order), and Ŝ_{p,b} denotes a target value(e.g., the target values of the spectrum generation parameter). y̅_̅{̅b̅}̅(I, L, μ, S_{p,b}′) refers to the photon counting estimation corresponding to each set of the plurality of sets of photon counting data; y_{b}, cali (I, L, μ, S_{p,b}′) refers to the photon counting subset corresponding to each set of the plurality of sets of photon counting data obtained under (I, L, μ); I denotes the tube current, μ denotes the attenuation coefficient of the phantom, L denotes the thickness of the phantom.

In some embodiments, the processor may iteratively update the initial values of the model parameters under different pulse pileup orders for multiple rounds based on the target optimization function. In each iterative update, the initial values of the model parameters after the current iterative update may be determined based on the value of the target optimization function, and whether the termination condition may be determined.

In some embodiments, in response to determining that the current iterative update does not satisfy the termination condition, the processor may determine, based on the updated values of the model parameters of the current iterative update, updated photon counting estimation corresponding to another set of the plurality of sets of photon counting data to be substituted into the target optimization function for the next iteration.

In some embodiments, in response to determining that the termination condition is satisfied, the processor may determine the updated values of the model parameters after the current iterative update as the target values of the model parameters.

In some embodiments, the termination condition may include that the value of the target optimization function is minimum, a count of iterations reaches a count threshold, the value of the target optimization function converges, etc. The count threshold may be a system default value, an experience value, an artificially preset value, or the like, or any combination thereof, and may be set according to actual needs.

In some embodiments of the present disclosure, the final value of the model parameter may be determined using an iterative update algorithm, which can effectively improve the calibration accuracy.

It should be noted that the foregoing descriptions of the spectrum model are for the purpose of example and illustration only, and do not limit the scope of application of the present disclosure. For those skilled in the art, various corrections and changes can be made to the contents related to the spectrum model under the guidance of the present disclosure. However, these corrections and changes remain within the scope of the present disclosure.

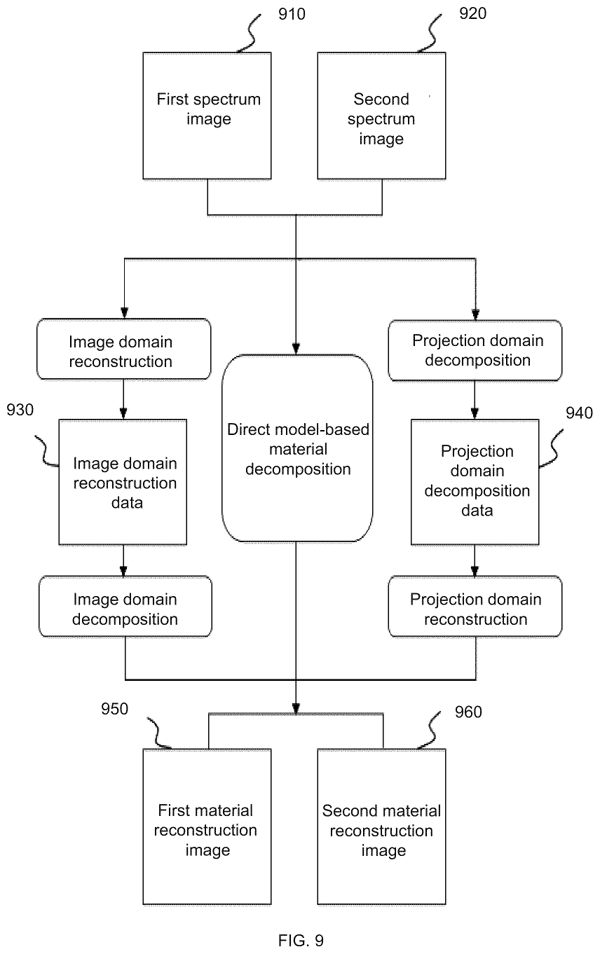

FIG. 9 is a schematic diagram illustrating exemplary basis material decomposition according to some embodiments of the present disclosure. As shown in FIG. 9, the basis material decomposition may include three manners, image domain decomposition, projection domain decomposition, and direct model-based material decomposition.

Processing of an image domain may include reconstruction and then decomposition. As for the image domain decomposition, a first spectrum image 910 (spectrum #1, i.e., first photon counting data) and a second spectrum image 920 (spectrum #2, i.e., second photon counting data) may be obtained. Image domain reconstruction data 930 may be obtained through image domain reconstruction based on the first spectrum image 910 and the second spectrum image 920. Decomposition data, i.e., first material reconstruction image 950 and a second material reconstruction image 960, may be obtained through the image domain decomposition based on the image domain reconstruction data 930.

Processing of a projection domain may include decomposition and then reconstruction. As for the projection domain decomposition, the first spectrum image 910 and the second spectrum image 920 may be obtained. Projection domain decomposition data 940, i.e., a first material line integral and a second material line integral, may be obtained through the projection domain decomposition based on the first spectrum image 910 and the second spectrum image 920. Reconstructed data, i.e., the first material reconstruction image 950 and the second material reconstruction image 960 may be obtained through projection domain reconstruction based on the projection domain decomposition data 940.

As for the direct model-based material decomposition, the first spectrum image 910 and the second spectrum image 920 may be obtained. The decomposition data, i.e., the first material reconstruction image 950 and the second material reconstruction image 960, may be obtained through a trained decomposition model based on the first spectrum image 910 and the second spectrum image 920.

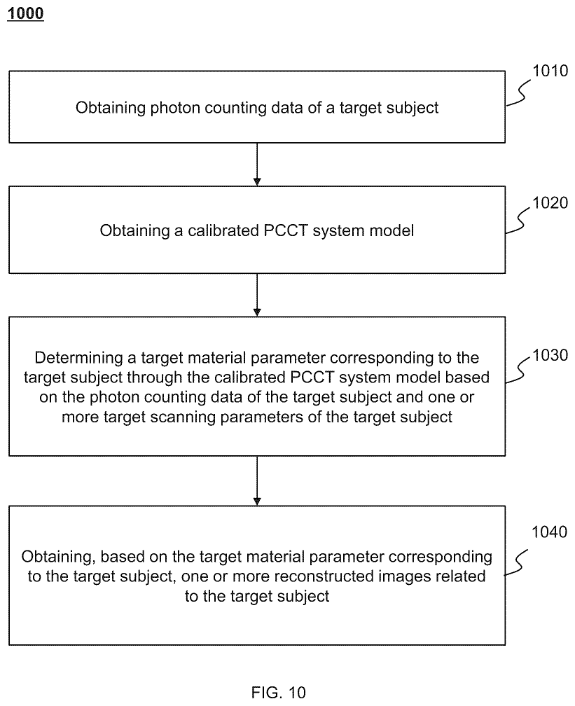

FIG. 10 is flowchart illustrating an exemplary process for image reconstruction according to some embodiments of the present disclosure. In some embodiments, process 1000 may be performed by the processing device 150. For example, process 1000 may be stored in a storage device (e.g., the storage device 110-4) in the form of a program or instruction, and when the processing device executes the program or instruction, the process 1000 may be implemented. The schematic diagram illustrating operation of the process 1000 below is illustrative. In some embodiments, the process may be completed by one or more additional operations not described and/or one or more operations not discussed. As shown in FIG. 10, the process 1000 of the image reconstruction method may include the following operations. In addition, the order of the operations of the process 1000 illustrated in FIG. 10 and described below is non-limiting.

In 1010, obtaining photon counting data of a target subject. In some embodiments, the photon counting data may be obtained by scanning the target subject based on one or more target scanning parameters using a PCCT device.

The target subject may be a subject to be scanned, e.g., a human body, etc.

In some embodiments, after a processor controls a radiation source (e.g., an X-ray source) of the PCCT device to scan the target subject, a detecting device (e.g., a PCD) of the PCCT device may generate corresponding photon counting data (i.e., the photon counting measurements), and the processor may obtain the photon counting data of the target subject In some embodiments, the photon counting data may be obtained based on a simulation algorithm, e.g., a Monte Carlo simulation algorithm.

More descriptions regarding the scanning parameters and the photon counting data may be found in FIG. 2 and FIG. 3 and related descriptions thereof.

In 1020, obtaining a calibrated PCCT system model. The calibrated PCCT system model may characterize or provide a mapping relationship between photon counting data, one or more scanning parameters (e.g., the tube current, the tube voltage, or the like, or a combination thereof) and material parameters of one or more reference materials (also referred to as reference material parameters).

A reference material refers to a base substance that is used to represent a subject (e.g., the target subject). For example, the target subject may be considered as a mixture of at least two different base substances (i.e., the base substances), and the at least two different base substances may also be referred to as the reference materials.

In some embodiments, the processor may obtain a plurality of sets of photon counting data by scanning a plurality of phantoms with different phantom parameters based on different scanning parameters using a PCCT device; and determine model parameters of a PCCT system model by calibrating the PCCT system model based on the scanning parameters corresponding to each set of the plurality of sets of photon counting data and the phantom parameters to obtain the calibrated PCCT system model. The calibrated PCCT system model may be obtained through the calibration process of the PCCT system model described in some embodiments of the present disclosure. More descriptions regarding calibrating the PCCT system model may be found in FIGS. 2 and 7B and related descriptions thereof.

In 1030, determining a target material parameter corresponding to the target subject through the calibrated PCCT system model based on the photon counting data of the target subject and the target scanning parameters of the target subject.

The target material parameter corresponding to the target subject may be related to one or more material parameters of one or more reference materials corresponding to the target subject. The reference materials corresponding to the target subject refer to base substances that are used to represent the target subject.

The types of reference materials corresponding to the target subject may be a default setting of the system 100 or set by an operator. For example, the reference materials corresponding to the target subject may include iodine, gadolinium, aluminum, etc.

In some embodiments, the target material parameter corresponding to the target subject may include the target thickness of each of the reference materials corresponding to the target subject.

In some embodiments, the processor may determine, based on the photon counting data of the target subject, the target scanning parameters of the target subject, and the attenuation coefficient of each of the reference materials corresponding to the target subject, the target thickness of each of the reference materials corresponding to the target subject through the calibrated PCCT system model.

In some embodiments, the processor may determine the target thicknesses of each of the reference materials corresponding to the target subject by inputting the photon counting data of the target subject, the one or more target scanning parameters of the target subject, and the attenuation coefficient of each of the reference materials corresponding to the target subject into the calibrated PCCT system model.

In some embodiments, the target material parameter corresponding to the target subject may include a material line integral of each of the reference materials corresponding to the target subject. The material line integral of a reference material corresponding to the target subject refers to data required for reconstructing a projection domain image of the target subject.

In some embodiments, the processor may determine, based on the target thickness of a reference material corresponding to the target subject and the attenuation coefficient of the reference material corresponding to the target subject, the material line integral of the reference material corresponding to the target subject. For example, a product of the target thickness of the reference material corresponding to the target subject and the attenuation coefficient of the reference material corresponding to the target subject may be determined as the material line integral of the reference material corresponding to the target subject.

More descriptions regarding determining the target thickness of the reference material corresponding to the target subject may be found in elsewhere the present disclosure.

In some embodiments, the processor may determine, based on the set of photon counting data, the target scanning parameter of the subject, a target material line integral corresponding to the target subject through the calibrated PCCT system model. The processor may determine, based on the target material line integral corresponding to the target subject, material line integrals of the one or more reference materials corresponding to the target subject. For example, the processor may decompose the target material line integral corresponding to the target subject into the material line integrals of the one or more reference materials corresponding to the target subject.

In some embodiments, the processor may input the photon counting data of the target subject into the calibrated PCCT system model, and determine, based on a correlation between the photon counting data and the material parameters of the reference materials corresponding to the target subject provided by the calibrated PCCT system model, the target material parameter (e.g., the thickness, the material line integral) corresponding to the target subject.

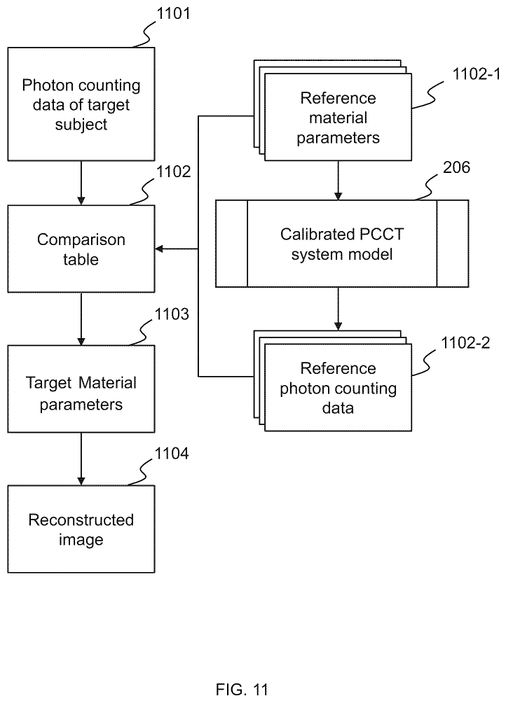

In some embodiments, the processor may construct, based on the calibrated PCCT system model, a comparison table containing a mapping relationship between the material parameter of each of one or more reference materials (e.g., the material thickness and/or the material line integral), photon counting data, and the scanning parameters. The processor may determine, based on the comparison table, the photon counting data of the target subject, and the scanning parameters of the target subject, the target material parameter (e.g., the target thicknesses and/or the target material line integral) of each of the reference materials corresponding to the target subject. More descriptions regarding determining, based on the comparison table, the target thicknesses and/or the target material line integrals of the reference materials corresponding to the target subject may be found in FIG. 11 and related descriptions thereof.

In some embodiments, after the photon counting data of the target subject are input into the calibrated PCCT system model, the processor may output, based on the spectrum model corresponding to each energy bin, the target material parameter (e.g., the target thickness, the target material line integral) corresponding to each energy bin.

In some embodiments, the processor may also optimize the output target material parameter (e.g., the target thickness, the target material line integral) corresponding to each energy bin through an MLEM algorithm.

In some embodiments, after the photon counting data of the target subject is input into the calibrated PCCT system model, the processor may output, based on the spectrum model corresponding to each energy bin, a material parameter of the reference material corresponding to each energy bin.

In 1040, obtaining, based on the target material parameter corresponding to the target subject, one or more reconstructed images related to the target subject.

In some embodiments, the processor may determine, based on the target material parameter corresponding to each energy bin, projection data corresponding the each energy bin (i.e., a Bin projection image), generate a virtual monoenergetic image (i.e., a VMI projection image) by combing the projection data corresponding the each energy bin, and obtain final reconstructed images based on the Bin projection image and the VMI projection image.

In some embodiments, the reconstructed images may include one or more material decomposition images.

In some embodiments, the processor may obtain, based on the target material parameter corresponding to the target subject, the material decomposition images of the reference materials corresponding to the target subject.

The material decomposition images may include qualitative and quantitative information about the tissue compositions of the target subject. In some embodiments, the material decomposition images may include an iodine-based image, a water-based image, a virtual plain scan image, an edema image, a fat content image, or the like, or a combination thereof.

The material decomposition image corresponding to a reference material may be determined based on the target material parameter determined in operation 1030. For example, the material line integral corresponding to each energy bin may be determined by multiplying the target thickness of the reference material corresponding to each energy bin and the attenuation coefficient of the reference material. The material decomposition image corresponding to the reference material may be reconstructed based on a combination of the material line integrals of the reference material corresponding to all the energy bins. For example, the material decomposition image may be reconstructed based on the material line integrals of the reference material corresponding to all the energy bins according to an image reconstruction algorithm, e.g., a FBP algorithm.

In some embodiments, the target material parameter determined in operation 1030 based on the calibrated PCCT system model may include a total material line integral (i.e., the target material line integral). The processor may determine the material decomposition images corresponding to at least two reference materials based on the total material line integral. For example, the total material line integral may be denoted as a third matrix. The third matrix may be decomposed into two matrixes (e.g., a first matrix and a second matrix). The first matrix may include elements each of which denotes an attenuation coefficient of one of the at least two reference materials. The first matrix may be a default setting of the system 100 or set by an operator. In other words, the at least two reference materials may be specified by the operator. The second matrix may include elements each of which denotes a thickness of one of the at least two reference materials. A material line integral corresponding to each of the at least two reference materials may be determined based on elements in the first matrix and the second matrix. A material decomposition image corresponding to one of the at least two reference materials may be determined based on the material line integral corresponding to the one of the at least two reference materials according to an image reconstruction algorithm.

In some embodiments, the attenuation coefficients of the reference materials corresponding to the target subject and the attenuation coefficient of the target subject be expressed by a formula (37):

$$μ ( E ) = {μ}_{1} ( E ) {c}_{1} + {μ}_{2} ( E ) {c}_{2} , \qquad ( 37 )$$

wherein μ(E) denotes the attenuation coefficient of each voxel in the target subject; μ₁(E) and μ₂(E) denote the attenuation coefficients of a base substance 1 (also referred to as reference material 1) and a base substance 2 (also referred to as reference material 2), respectively, and μ₁(E) and μ₂(E) are known; c₁ and c₂ denote mass densities of the base substance 1 and the base substance 2, respectively, and c₁ and c₂ may be estimated through material decomposition. Each of c₁ and c₂ may be denoted as a matrix. The matrix representing c₁ or c₂ may include multiple elements each of which corresponds to a voxel in the target subject and represents a relationship between the attenuation coefficient of the base substance 1 or the base substance 2 and the attenuation coefficient of the voxel in the target subject. The matrix representing c₁ or c₂ may also referred to as a material decomposition image. It should be noted that the two reference materials may be merely for illustration, the target subject may be denoted by more than two reference materials.

In some embodiments, the reconstructed images may include a virtual image.

In some embodiments, the virtual image may include the VMI (i.e., the VMI projection image). The VMI projection image refers to a CT image obtained by simulating monochromatic radiation rays of arbitrary energy.

In some embodiments, as the attenuation coefficient is a function related to energy, according to formula (36), the attenuation coefficient of the target subject at a specific energy level (keV) may be denoted by the attenuation coefficients of the reference materials at the specific energy level (keV) and the material decomposition images corresponding to the reference materials. In other words, a virtual image at a specific energy level (keV) may be determined based on the attenuation coefficient of the target subject at the specific energy level (keV).

In some embodiments, the virtual image at a specific energy level (keV) of the target subject may be obtained by weighting and summing the material decomposition images corresponding to the reference materials. The weighting values corresponding to the reference materials may be determined based on the attenuation coefficients of the reference materials at the specific energy level (keV). Another example, the virtual image at a specific energy level (keV) of the target subject may include multiple pixels representing CT values of different portions of the target subject. The processor may determine the attenuation coefficients of different portions of the target subject according to formula (37) and determine the CT values of different portions of the target subject according to formula (38) as followings:

$$E = 1000 \times \frac{[ μ ( E ) - {μ}_{1} ( E ) ]}{{μ}_{1} ( E )} , \qquad ( 38 )$$

where E denotes the CT values at the specific energy level (keV) of different portion of the target subject, and μ₁(E) denotes the attenuation coefficient, at the specific energy level (keV), of a first reference material (e.g., water) among the reference material corresponding to the target subject. E may be denoted as an image, i.e., the virtual image at a specific energy level (keV) of the target subject. In some embodiments, the photon counting data of the target subject may include a plurality of subsets of photon counting data corresponding to different energy bins, and the target material parameter corresponding to the target subject may include the target material line integrals of the reference materials corresponding to the target subject. Correspondingly, the processor may determine, based on the plurality of subsets of photon counting data, the target material line integrals of the reference materials corresponding to the target subject under different energy bins; determine, based on the target material line integrals of the reference materials corresponding to the target subject under different energy bins, projection data of the different energy bins; and obtain, based on the projection data of the different energy bins, the material decomposition images of the reference material corresponding to the target subject.

The descriptions regarding determining the target material line integrals of the reference materials corresponding to the target subject may be found in the previous descriptions.

In some embodiments, the processor may perform projection domain decomposition based on the target material line integrals of the reference materials corresponding to the target subject under different energy bins to obtain the projection data of the different energy bins, i.e., the Bin projection images. The processor may synthesize the projection data of the different energy bins to reconstruct the material decomposition images of the reference materials corresponding to the target subject.

In some embodiments, the processor may reconstruct, based on high-energy photon counting data and low-energy photon counting data acquired by scanning the target subject at a first tube current and a second tube current, a first reconstructed image and a second reconstructed image; and obtain, based on the first reconstructed image and the second reconstructed image, the virtual image of the target. For example, the processor may weight and sum the first reconstructed image and the second reconstructed image to obtain an intermediate image. The virtual image may be obtained based on a difference between the intermediate image and a material decomposition image.

In some embodiments, the processor may reconstruct, based on the high-energy photon counting data and the low-energy photon counting data, the first reconstructed image and the second reconstructed image in any feasible way. For example, the processor may process the photon counting data using an iterative reconstruction (IR) algorithm to perform image reconstruction.

In some embodiments, the processor may perform weighted fusion on the first reconstructed image and the second reconstructed image, and subtract the material decomposition image from a fused reconstructed image obtained by the weighted fusion to obtain the virtual image. A weight for the weighted fusion may include a system default value, an experience value, an artificially preset value, or the like, or any combination thereof, which is set according to actual needs and is not limited in the present disclosure.

In some embodiments of the present disclosure, after the photon counting data of the target subject is obtained, the material parameters of the reference materials corresponding to the target subject may be more conveniently and quickly obtained through the PCCT system model calibrated by the calibration method for the PCCT system model according to some embodiments of the present disclosure. The accuracy of the obtained material parameters of the reference materials may be higher, which helps to eliminate artifacts caused by energy spectrum distortion and pulse pileup, and improve the imaging quality.

It should be noted that the foregoing description of the process 1000 is intended to be exemplary and illustrative only, and does not limit the scope of the present disclosure. For those skilled in the art, various corrections and changes may be made to the operations of the process 1000 under the guidance of the present disclosure. However, these corrections and changes remain within the scope of the present disclosure.

FIG. 11 is a schematic diagram illustrating an exemplary process for image reconstruction o according to some other embodiments of the present disclosure. In some embodiments, process 1100 may be performed by the processing device 150. For example, process 1100 may be stored in a storage device (e.g., the storage device 110-4) in the form of a program or instruction, and when the processing device executes the program or instruction, the process 1100 may be implemented. The schematic diagram illustrating operation of the process 1100 below is illustrative. In some embodiments, the process may be completed by one or more additional operations not described and/or one or more operations not discussed. As shown in FIG. 11, the process 1100 of the image reconstruction method may include the following operations. In addition, the order of the operations of the process 1100 illustrated in FIG. 11 and described below is non-limiting.

Photon counting data 1101 of a target subject may be obtained.

More descriptions regarding the target subject and photon counting data may be found in FIG. 10 and related descriptions thereof.

Target material parameters 1103 of one or more reference materials corresponding to the target subject may be determined through a comparison table 1102 based on the photon counting data 1101 of the target subject.

In some embodiments, the comparison table 1102 may include correspondence relationships between a plurality of reference material parameters 1102-1, reference scanning parameters, and reference photon count data 1102-2. The comparison table 1102 may provide multiple groups of reference data. Each group of the multiple groups of reference data may include one or more reference scanning parameters, reference material parameters, and reference photon counting data corresponding to the one or more reference scanning parameters and the reference material parameters.

The comparison table 1102 may be determined based on the calibrated PCCT system model as described elsewhere in the present disclosure. For example, for one group of reference data, the reference photon counting data corresponding to the one or more reference scanning parameters and the reference material parameters may be determined by inputting the one or more reference scanning parameters and the reference material parameters into the calibrated PCCT system model. As another example, the processor may input the reference photon count data 1102-2 and the reference scanning parameters into the calibrated PCCT system model 206. The calibrated PCCT system model 206 may output the corresponding reference material parameters 1102-1 based on the correlation between the reference photon counting data, the reference scanning parameters, and the reference material parameters.

The reference material parameters 1102-1 refer to the material parameters of reference materials. In some embodiments, the processor may obtain a plurality of arbitrary material parameters as the reference material parameters 1102-1 from experimental measurements, computational data, historical databases, etc. For example, a plurality of thicknesses of different reference materials (not limited to PMMA/AL mentioned above) may be obtained, an attenuation coefficient corresponding to each of the different reference materials may be, and then the plurality of reference material line integrals of the reference materials may be determined based on the thicknesses and the attenuation coefficients of the reference materials.

The reference photon counting data 1102-2 refers to photon counting data used to provide a reference in the comparison table. Each of the reference photon counting data 1102-2 may correspond to one of the reference material parameters 1102-1 in the comparison table.

In some embodiments, the calibrated PCCT system model may be obtained by the calibration process for the PCCT system model as described in the present disclosure. See, FIGS. 2 and 7B.

In some embodiments, at least a portion of the multiple groups of reference data may be obtained based on scans of the PCCT device. For example, the processor may obtain a plurality of sets of reference photon counting data by scanning a plurality of phantoms with different reference phantom parameters based on different reference scanning parameters.

In some embodiments, the processor may look up the comparison table based on the photon counting data of the target subject and the scanning parameter of the target subject to determine reference photon counting data that is the same as or similar to the photon counting data of the target subject, and determine the target material parameter of the reference material corresponding to the target subject based on the reference material parameter corresponding to the reference photon counting data.

For example, the processor may designate the reference material parameter corresponding to the reference photon counting data as the target material parameter of the reference material corresponding to the target subject.

In some embodiments, the processor may determine a similarity degree between the reference photon counting data and the photon counting data of the target subject. If the similarity degree between the reference photon counting data and the photon counting data of the target subject exceeds a similarity threshold (e.g., 90%, 95%, etc.), the reference photon counting data may be considered to be the same as or similar to the photon counting data of the target subject. The similarity threshold may be a default setting of the system 100 or set by an operator.

In some embodiments, the processor may determine, based on the photon counting data of the target subject and the scanning parameter of the target subject, first photon counting data and second photon counting data from the comparison table; determine, based on the first photon counting data and the second photon counting data, a first thickness and a second thickness of the reference materials corresponding to the target subject, respectively; and determine, based on the first thickness and the second thickness, the target thicknesses of the reference materials corresponding to the target subject through interpolation processing.

In some embodiments, the processor may look up, based on the photon counting data of the target subject, two reference photon count data that are similar to or same as the photon counting data of the target subject in the comparison table as the first photon counting data and second photon counting data.

In some embodiments, the reference materials corresponding to the first photon counting data and the second photon counting data may be the same. The processor may perform interpolation processing in any feasible way based on the first thickness and the second thickness to determine the thicknesses of the reference materials corresponding to the target subject. A manner of interpolation processing may include but is not limited to two-dimensional interpolation, Lagrange interpolation, Newton interpolation, etc.

Image reconstruction may be performed based on the material parameters 1103 to generate a reconstructed image 1104.

For example, the processor may obtain the reconstructed image 1104 by performing image reconstruction based on a material line integral. The implementation of the operation may be found in the relevant operations and related descriptions thereof in FIG. 10.

In some embodiments of the present disclosure, the correspondences between the plurality of reference material parameters (e.g., material line integrals) and the reference photon counting data corresponding to the plurality of reference material line integrals may be established in advance, and the data may be obtained by looking up the table when necessary, reducing the reliance on the real-time operation of the PCCT system model. The preset lookup table may include various combinations of material parameters (e.g., material line integrals) and photon counting data that the target subject may have, allowing for more accurate matching when the photon counting data of the target object is compared with the reference photon counting data in the preset lookup table. The reference photon counting data may be determined based on the reference material parameters (e.g., material line integrals) and the calibrated PCCT system model when the preset lookup table is established, facilitating the establishment of the correspondence between the plurality of reference material line integrals and the reference photon counting data corresponding to the plurality of reference material line integrals. The target material parameters (e.g., material line integrals) may be quickly determined through the preset lookup table based on the photon counting data of the target subject, improving the imaging efficiency of the target object. Image reconstruction may be performed based on the reference material line integrals corresponding to the reference photon counting data that matches the photon counting data of the target subject, so that artifacts caused by energy spectrum distortion and pulse pileup may be eliminated, thus enhancing the imaging quality.

It should be noted that the foregoing descriptions of the steps in the method of image reconstruction are for the purpose of exemplification and illustration only, and do not limit the scope of application of the present disclosure. For those skilled in the art, various corrections and changes may be made to the operations of the image reconstruction method under the guidance of the present disclosure. However, these corrections and changes remain within the scope of the present disclosure.

Some embodiments of the present disclosure further provide a calibration device for a PCCT system model. The calibration device may comprise at least one processor and at least one storage device. The at least one storage device may be configured to store computer instructions. The at least one processor may be configured to execute at least part of the computer instructions to implement the calibration method of the PCCT system model described in any of the above embodiments

One or more embodiments of the present disclosure further provide a medical device comprising a processor configured to implement the calibration method for the PCCT system model as described in any of the above embodiments.

One or more embodiments of the present disclosure further provide a non-transitory computer-readable storage medium comprising storage instructions that, when read by a computer, may direct the computer to perform the calibration method for the PCCT system model as described in any of the above embodiments.

In order to further illustrate the effectiveness of the technical solution disclosed in the present disclosure, exemplary illustrations are shown of effect diagrams in the process of image reconstruction using the calibrated PCCT system model by the calibration method for the PCCT system model in some embodiments of the present disclosure. The effect that may be achieved by the technical solution is illustrated by comparing the effect with an effect diagram using a simple empirical model.

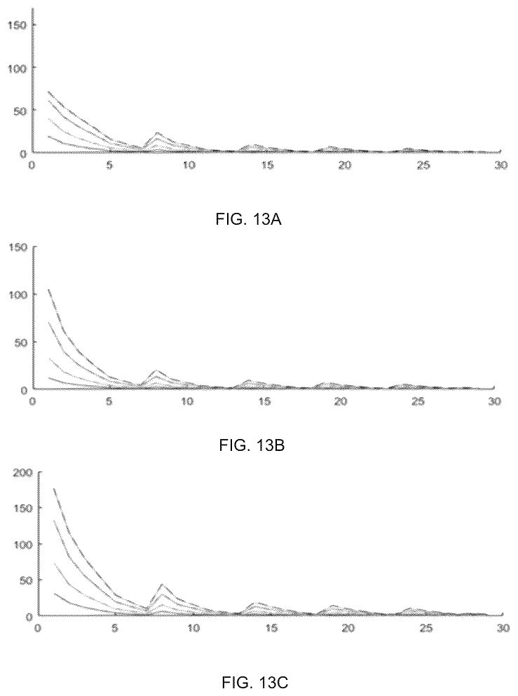

FIGS. 13A-13C are comparison plots of photon counting data obtained by calculations using a calibrated PCCT system model and photon counting data obtained by a validation test according to some embodiments of the present disclosure. FIG. 13a is a comparison plot illustrating calculated photon counting data corresponding to a low-energy bin (LE Bin) versus photon counting data obtained by a validation test. FIG. 13b is a comparison plot illustrating calculated photon counting data corresponding to a high-energy bin (HE Bin) versus photon counting data obtained by a validation test. FIG. 13c is a comparison plot illustrating calculated photon counting data after merging (All Bin) a low-energy bin and a high-energy bin versus photon counting data obtained by a validation test. Parameters used in the validation test may include that the scanning device is 1 cm PCCT, a mode of the scanning device is a human capital mode, a peak voltage of the scanning device is 140 kVp, a tube current of the scanning device during scanning includes 50 mA, 100 mA, 150 mA and 200 mA, critical setting is null, packing parameters are [30, 65, 255], a scanning mode is static, a start angle of scanning is 180 degrees, a viewing mode is 2400 times per second, which is 2 seconds in total, focus setting is USFS (standard single focus) and small focus. In FIGS. 13A-13C, a horizontal coordinate represents a count of test sets tested based on different material thicknesses, and a vertical coordinate represents photon counting data in Mcps (millions of counts per second). In each of FIGS. 13A-13C, data corresponding to scanning tube current of 200 mA, 150 mA, 100 mA, 50 mA are shown from top to bottom. A solid line represents actual photon count data generated by a detector based on a certain tube current and scanning a specific phantom, while a dotted line represents a preset material line integral corresponding to the same tube current and the same phantom (if there are other parameters, e.g., the incident count rate, the other parameters may be consistent with parameters of the validation test). The calculated photon counting data obtained by the calibrated PCCT system model may be shown in some embodiments of the present disclosure. As can be seen from FIGS. 13A-13C, the dotted line may overlap with the solid line to a high degree, the PCCT system model may have a high degree of direct fitting to the actual data with a small error, and the PCCT system model may accurately describe the energy response of incident photons.

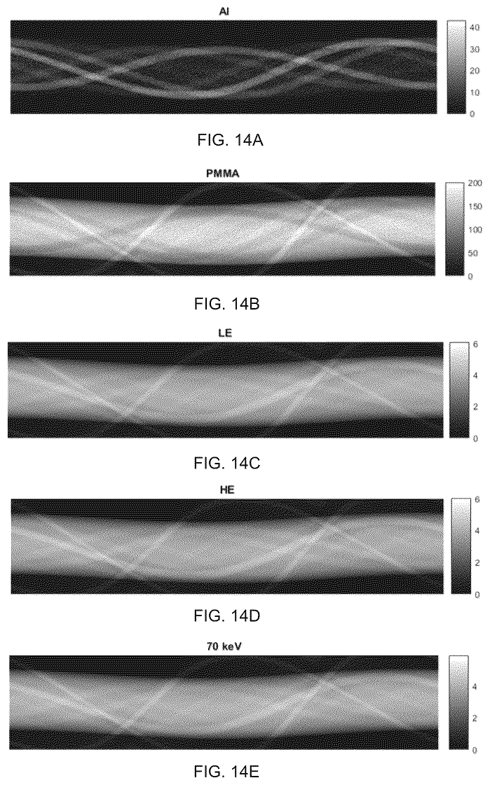

FIGS. 14A-14E are schematic diagrams illustrating a target material line integral obtained by an image reconstruction method according to some embodiments of the present disclosure. After a target subject is scanned, the target material line integral may be determined as shown in FIGS. 14A-14E by the image reconstruction method according to some embodiments of the present disclosure based on the photon counting data of the target object. FIG. 14A shows a material line integral of Al, and FIG. 14B shows a material line integral of PMMA. A material line integral corresponding to a low-energy bin (LE) of FIG. 14C, a material line integral corresponding to high-energy bins (HE) of FIG. 14D, and a material line integral corresponding to photon energy of 70 keV in FIG. 14E may be obtained, respectively, by merging material line integrals of selected energy bins.

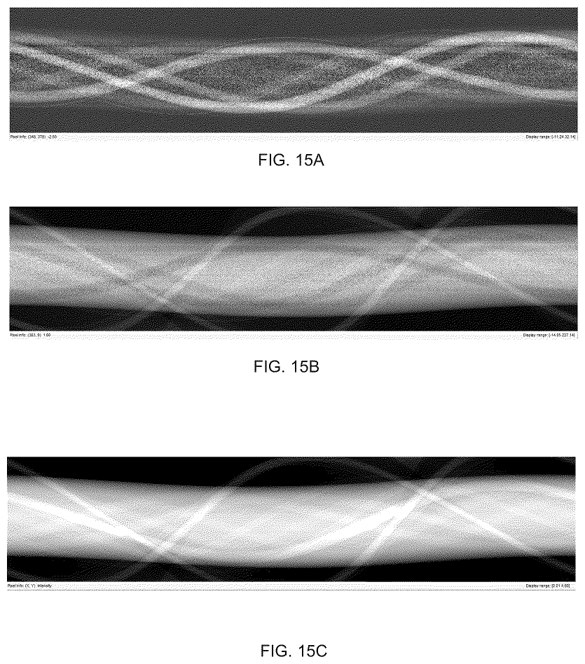

FIGS. 15A-15C are schematic diagrams illustrating material line integrals obtained through a simple empirical model. FIG. 15A shows a material line integral of Al, FIG. 15B shows a material line integral of PMMA, and FIG. 15C shows a material line integral corresponding to a tube current of 50 mA. Compared with FIGS. 15A-15C, the target material line integrals (FIGS. 14A-14C) obtained by the image reconstruction method according to some embodiments of the present disclosure may exhibit significantly reduced artifacts, richer image details, and higher image quality.

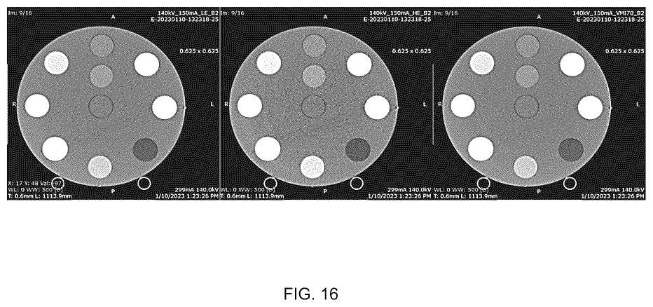

FIG. 16 is a schematic diagram illustrating a reconstructed image obtained using a target material line integral corresponding to a tube current of 150 mA of an image reconstruction method according to some embodiments of the present disclosure. The reconstruction method may be based on lookup table (LUT) and filtered back projection (FBP) techniques.

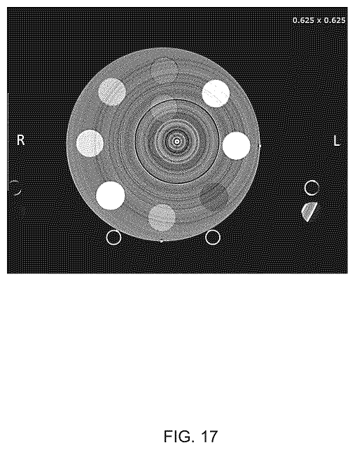

FIG. 17 is schematic diagrams illustrating reconstructed images obtained by reconstruction of material line integrals corresponding to 50 mA tube current obtained through a simple empirical model. Compared with FIG. 17, the reconstructed image (FIG. 16) obtained by the image reconstruction method shown according to some embodiments of the present disclosure may exhibit significantly reduced ring artifacts and higher image quality.

Possible beneficial effects of embodiments of the present disclosure include, but are not limited to the following content.

(1) In some embodiments of the present disclosure, the PCCT system model is proposed, and parameter fitting is performed on the PCCT system model through the photon counting data obtained by scanning the plurality of phantoms, the preset scanning parameters of the scanning, and the preset material line integral corresponding to the plurality of phantoms, so the calibration of the PCCT system model is completed, and the PCCT system model considering the X-ray source intensity and spectrum, imaging object filtration, and the features of the detector can be obtained. The X-ray source intensity and spectrum can be reflected by the preset scanning parameters, the imaging object filtration can be reflected by the material line integrals corresponding to the plurality of phantoms, and the features of the detector can be reflected by the spectral measurements obtained under the preset scanning parameters and the preset material line integral. Therefore, the resulting PCCT system model is the physical model based on the photon transmission path, has a more precise energy response to the incident photons, and achieves a higher degree of accuracy and smaller error in fitting with actual data. The PCCT system model can accurately describe the energy response of the incident photons and can be used for image reconstruction of the target object, thereby generating more accurate reconstructed images.

(2) In some embodiments of the present disclosure, the plurality of sets of spectral measurements are obtained by performing multiple scans of the plurality of phantoms, allowing for obtaining as much data as possible for calibration, reducing the impact of data noise and chance events that may introduce errors, and improving the accuracy of calibration results. In addition, the obtained calibration results can comprehensively cover a wide range of the scanning parameters, the plurality of phantoms, and the target subject, thereby being applicable in a wider range.

(3) In some embodiments of the present disclosure, the PCCT system model is composed of the energy deposition sub-model, the energy resolution sub-model, the charge sharing sub-model, the pulse pileup sub-model, and the energy binning sub-model by cascading. The detector response is parameterized by the corresponding sub-models at each stage, so that the obtained PCCT system model is the physical model based on the photon transmission path, which can gradually eliminate or partially eliminate the effects of energy deposition, energy resolution, charge sharing, pulse pileup, and energy binning phenomena on photon counting, and is conducive to improving the accuracy of the PCCT system model.

(4) In some embodiments of the present disclosure, important factors affecting photon counting such as the incident count rate, the tube current, the attenuation coefficient, and the material thickness combination that affect photon counting are found by scanning the plurality of phantoms and combining the experimental results with the plurality of phantoms and the scanning parameters for scanning. These important factors are included as at least one parameter of the spectrum model of formula (4). By using the spectrum model, a plurality of model parameters affecting photon counting in the photon transmission path process are compressed, which helps to obtain sparser spectrum parameters. Through the spectrum model of the formula (4), the PCCT system model is not only simplified, but also different influences that pulse pileup effects of different orders may have on photon counting are also considered, so that the spectrum model includes energy response spectra at different pulse pileup orders can be formed, and the resulting PCCT system model can eliminate counting errors caused by pulse pileup as much as possible.

(5) In some embodiments of the present disclosure, the spectrum parameters can be calculated and determined by substituting the photon counting data, the scanning parameters, and the material line integrals into the spectrum model, thereby obtaining the spectrum parameters more conveniently and quickly.

(6) In some embodiments of the present disclosure, the non-pileup energy response spectrum at the pulse pileup order 0 is determined by testing at the low tube current, and the model parameters determined by the calibration method for the PCCT system model are optimized through the optimized algorithm based on the non-pileup energy response spectrum, thereby improving the accuracy of the spectrum parameters of the PCT system model, and optimizing the energy spectrum response of the PCCT system model.

(7) In some embodiments of the present disclosure, after the photon counting data of the target object is obtained, the material line integral corresponding to the target object can be more conveniently and quickly obtained by the PCCT system model calibrated by the calibration method for the PCCT system model according to some embodiments of the present disclosure, and the accuracy of the obtained material line integrals is high, so that the artifacts caused by spectrum distortion and pulse pileup can be eliminated, thereby improving the imaging quality.

(8) In some embodiments of the present disclosure, the correspondence between the plurality of reference material line integrals and the reference photon counting data corresponding to the plurality of reference material line integrals are pre-established, and the table can be referenced in need to reduce reliance on the real-time operation of the PCCT system model. The comparison table can include as many combinations of the material line integrals and the photon counting data as possible for the target subject, ensuring more accurate matching when the photon counting data of the target subject is compared with the reference photon counting data in the comparison table. The reference photon counting data are determined based on the reference material line integrals and the calibrated PCCT system model, making it more convenient and quickly to obtain the correspondence between the plurality of reference material line integrals and the reference photon counting data corresponding to the plurality of reference material line integrals. The target material line integral can be quickly determined through the comparison table based on the photon counting data of the target object, thereby improving the efficiency of imaging the target subject. In addition, image reconstruction based on the reference material line integrals corresponding to the reference photon counting data matching the photon counting data of the target object can effectively eliminate the artifacts caused by spectrum distortion and pulse pileup, thereby improving the imaging quality.

The basic concept has been described above. Obviously, for those skilled in the art, the above detailed disclosure is only an example, and does not constitute a limitation to the present disclosure. Although not expressly stated here, those skilled in the art may make various modifications, improvements and corrections to the present disclosure. Such modifications, improvements and corrections are suggested in this disclosure, so such modifications, improvements and corrections still belong to the spirit and scope of the exemplary embodiments of the present disclosure.

Meanwhile, the present disclosure uses specific words to describe the embodiments of the present disclosure. For example, “one embodiment”, “an embodiment”, and/or “some embodiments” refer to a certain feature, structure or characteristic related to at least one embodiment of the present disclosure. Therefore, it should be emphasized and noted that references to “one embodiment” or “an embodiment” or “an alternative embodiment” two or more times in different places in the present disclosure do not necessarily refer to the same embodiment. In addition, certain features, structures or characteristics in one or more embodiments of the present disclosure may be properly combined.

In addition, unless clearly stated in the claims, the sequence of processing elements and sequences described in the present disclosure, the use of counts and letters, or the use of other names are not used to limit the sequence of processes and methods in the present disclosure. While the foregoing disclosure has discussed by way of various examples some embodiments of the invention that are presently believed to be useful, it should be understood that such detail is for illustrative purposes only and that the appended claims are not limited to the disclosed embodiments, but rather, the claims are intended to cover all modifications and equivalent combinations that fall within the spirit and scope of the embodiments of the present disclosure. For example, although the implementation of various components described above may be embodied in a hardware device, it may also be implemented as a software only solution, e.g., an installation on an existing server or mobile device.

In the same way, it should be noted that in order to simplify the expression disclosed in this disclosure and help the understanding of one or more embodiments of the invention, in the foregoing description of the embodiments of the present disclosure, sometimes multiple features are combined into one embodiment, drawings or descriptions thereof. This method of disclosure does not, however, imply that the subject matter of the disclosure requires more features than are recited in the claims. Rather, claimed subject matter may lie in less than all features of a single foregoing disclosed embodiment.

In some embodiments, counts describing the quantity of components and attributes are used. It should be understood that such counts used in the description of the embodiments use the modifiers “about”, “approximately” or “substantially” in some examples. Unless otherwise stated, “about”, “approximately” or “substantially” indicates that the stated figure allows for a variation of ±20%. Accordingly, in some embodiments, the numerical parameters used in the disclosure and claims are approximations that can vary depending upon the desired characteristics of individual embodiments. In some embodiments, numerical parameters should consider the specified significant digits and adopt the general digit retention method. Although the numerical ranges and parameters used in some embodiments of the present disclosure to confirm the breadth of the range are approximations, in specific embodiments, such numerical values are set as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A system, comprising:
at least one processor and at least one storage device; wherein
the at least one storage device is configured to store computer instructions; and
the at least one processor is configured to communicate with the at least one storage device to direct, when the computer instructions are executed, the system to:
obtain a plurality of sets of photon counting data, each set of the plurality of sets of photon counting data being acquired by a photon counting computed tomography (PCCT) device scanning one of a plurality of phantoms according to one or more scanning parameters;
obtain a PCCT system model; and
calibrate the PCCT system model by determining, based on the plurality of sets of photon counting data, the one or more scanning parameters, and a parameter of the phantom, one or more model parameters of the PCCT system model.

2. The system of claim 1, wherein the model parameters are related to at least one procedure of X-ray generation, X-ray absorption, energy deposition, energy resolution, charge sharing, pulse pileup effect, or energy separation in a generation process of photon counting data.

3. The system of claim 2, wherein the PCCT system model includes a plurality of sub-models, and the plurality of sub-models include at least one of:
an energy deposition sub-model representing a relationship between the energy deposition and the photon counting data,
an energy resolution sub-model representing a relationship between the energy resolution and the photon counting data,
a charge sharing sub-model representing a relationship between the charge sharing and the photon counting data,
a pulse pileup sub-model representing a relationship between the pulse pileup and the photon counting data, or
an energy separation sub-model representing a relationship between the energy separation and the photon counting data.

4. The system of claim 1, wherein the model parameters include one or more groups, each group of the one or more groups of the model parameters corresponding to different pulse pileup orders under an energy bin.

5. The system of claim 4, wherein the calibrated PCCT system model includes one or more spectrum models each of which corresponds to one of one or more energy bins, each of the one or more spectrum models representing a mapping relationship between photon counting data under the energy bin, the scanning parameter, and a parameter of a reference material through a group of model parameters corresponding to the energy bin.

6. The system of claim 5, wherein calibrating the PCCT system model based on the plurality of sets of photon counting data including determining a group of model parameters corresponding to an energy bin according to operations includes:
for one of the different pulse pileup orders under the energy bin,
determining an initial value of a model parameter among the group of model parameters corresponding to the pulse pileup order; and
determining a target value of the model parameter based on the initial value of the model parameter corresponding to the pulse pileup order.

7. The system of claim 6, wherein the system performs an optimization of the model parameter based on the initial value of the model parameter corresponding to the pulse pileup order to determine the target value of the model parameter, and the optimization includes performing an iterative process including multiple iterations, in each iteration,
determining, based on a portion of a set of photon counting data under the energy bin, the parameter of the phantom and the one or more scanning parameters corresponding to the set of photon counting data, photon counting estimation through the spectrum model including the initial value of the model parameter;
constructing, based on an error between the portion of the set of the photon counting data under the energy bin and the photon counting estimation, a target optimization function;
updating the initial value of the model parameter based on the target optimization function to obtain an updated value of the model parameter that is designated as the initial value of the model parameter in a next iteration; and
in response to determining that a termination condition is satisfied, determining an updated value of the model parameter as the target value of the model parameter.

8. The system of claim 6, wherein the determining an initial value of a model parameter corresponding to the pulse pileup order includes:
determining, based on a reference set of photon counting data obtained under a scanning parameter satisfying a condition, a reference model parameter; and
determining, based on the reference model parameter, the initial value of the model parameter corresponding to the pulse pileup order.

9. The system of claim 8, wherein the determining, based on the reference model parameter, the initial value of the model parameter corresponding to the pulse pileup order includes:
in response to determining that the pulse pileup order is zero, determining the initial value of the model parameter corresponding to the pulse pileup order by normalizing the reference model parameter based on a reference tube current.

10. The calibration system of claim 8, wherein the determining, based on the reference model parameter, the initial value of the model parameter corresponding to the pulse pileup order includes:
in response to determining that the pulse pileup order is non-zero, determining an initial value of the model parameter corresponding to a pulse pileup order of zero by normalizing the reference model parameter based on a reference tube current; and
determining, based on the initial value of the model parameter corresponding to the pulse pileup order being of zero, the initial value of the model parameter corresponding to the pulse pileup order being of non-zero.

11. A system, comprising:
at least one processor and at least one storage device; wherein
the at least one storage device is configured to store computer instructions; and
the at least one processor is configured to communicate with the at least one storage device to direct, when the computer instructions are executed, the system to:
obtain a set of photon counting data acquired by a PCCT device scanning a target subject according to a target scanning parameter;
obtain a calibrated PCCT system model representing a mapping relationship between photon counting data, a scanning parameter, and one or more material parameters of one or more reference materials;
determine, based on the set of photon counting data and the target scanning parameter of the target subject, one or more target material parameters corresponding to the target subject via the calibrated PCCT system model; and
obtain, based on the one or more target material parameters corresponding to the subject, one or more reconstructed images related to the target subject.

12. The system of claim 11, wherein the determining, based on the set of photon counting data and the target scanning parameter of the target subject, the target material parameters corresponding to the target subject through the calibrated PCCT system model includes:
determining, based on the set of photon counting data, the target scanning parameter of the subject, and attenuation coefficients of the one or more reference materials corresponding to the target subject, target thicknesses of the one or more reference materials corresponding to the target subject through the calibrated PCCT system model.

13. The system of claim 12, wherein the obtaining, based on the target material parameters of the reference materials corresponding to the target subject, reconstructed images related to the target subject includes:
determining, based on the target thicknesses of the one or more reference materials, a material line integral of each of the reference materials corresponding to the target subject; and
generating, based on the material line integral of each of the reference materials corresponding to the target subject, a material decomposition image of each of the reference materials corresponding to the target subject.

14. The system of claim 11, wherein the determining, based on the set of photon counting data and the target scanning parameter of the target subject, the target material parameters corresponding to the target subject through the calibrated PCCT system model includes:
determining, based on the set of photon counting data, the target scanning parameter of the subject, and attenuation coefficients of the one or more reference materials corresponding to the target subject, a target material line integral corresponding to the target subject through the calibrated PCCT system model.

15. The system of claim 14, wherein the obtaining, based on the target material parameters corresponding to the subject, one or more reconstructed images related to the target subject includes:
determining, based on the target material line integral corresponding to the target subject, material line integrals of the one or more reference materials corresponding to the target subject; and
reconstructing a material decomposition image of the target subject based on one of the material line integrals of the one or more reference materials corresponding to the target subject.

16. The system of claim 11, wherein the determining, based on the set of photon counting data and the target scanning parameter of the target subject, the target material parameters corresponding to the target subject through the calibrated PCCT system model includes:
constructing, based on the calibrated PCCT system model, a comparison table; and
determining, based on the comparison table, the set of photon counting data, and the target scanning parameter of the target subject, the target material parameters corresponding to the target subject.

17. The system of claim 16, wherein the determining, based on the comparison table, the set of photon counting data, and the target scanning parameter of the target subject, the target material parameters corresponding to the target subject includes:
determining, based on the set of photon counting data and the target scanning parameter, first photon counting data and second photon counting data from the comparison table;
determining, based on the first photon counting data and the second photon counting data, a first material parameter and a second material parameter corresponding to the target subject, respectively; and
determining, based on the first material parameter and the second material parameter, a target material parameter corresponding to the target subject via interpolation processing.

18. The system of claim 11, wherein the determining, based on the set of photon counting data and the target scanning parameter of the target subject, the target material parameters corresponding to the target subject through the calibrated PCCT system model includes:
obtaining the target material parameters corresponding to the target subject by inputting the set of photon counting data and the target scanning parameter of the target subject into the calibrated PCCT system model.

19. The system of claim 11, wherein the obtaining a calibrated PCCT system model includes:
obtaining a plurality of sets of photon counting data, each set of the plurality of sets of photon counting data being acquired by the PCCT device scanning one of a plurality of phantoms according to one or more scanning parameters;
obtaining a PCCT system model; and
calibrating the PCCT system model by determining, based on the plurality of sets of photon counting data, the one or more scanning parameters, and a parameter of the phantom, one or more model parameters of the PCCT system model.

20. The system of claim 11, wherein the set of photon counting data includes a plurality of subsets of photon counting data corresponding to different energy bins; and
the obtaining, based on the one or more target material parameters corresponding to the subject, one or more reconstructed images related to the target subject includes:
determining, based on the plurality of subsets of photon counting data, a material line integral of each of the reference materials under the different energy bins;
determining, based on the material line integrals of the reference materials under the different energy bins, projection data of at least one energy bin; and
obtaining, based on the projection data of the at least one energy bin, a material decomposition image corresponding to the target subject.
